SEQUENCE LISTING (23 sequences)

SEQ ID NO: 1 - RNA, 2722 nt, Homo sapiens
caacaguccccaggcaucaccauucaagaugcauccagggguccuggcugccuuccucuucuugagcuggacucauugucgggcccugccccuucccagugguggugaugaagaugauuugucugaggaagaccuccaguuugcagagcgcuaccugagaucauacuaccauccuacaaaucucgcgggaauccugaaggagaaugcagcaagcuccaugacugagaggcuccgagaaaugcagucuuucuucggcuuagaggugacuggcaaacuugacgauaacaccuuagaugucaugaaaaagccaagaugcgggguuccugaugugggugaauacaauguuuucccucgaacucuuaaaugguccaaaaugaauuuaaccuacagaauugugaauuacaccccugauaugacucauucugaagucgaaaaggcauucaaaaaagccuucaaaguuugguccgauguaacuccucugaauuuuaccagacuucacgauggcauugcugacaucaugaucucuuuuggaauuaaggagcauggcgacuucuacccauuugaugggcccucuggccugcuggcucaugcuuuuccuccugggccaaauuauggaggagaugcccauuuugaugaugaugaaaccuggacaaguaguuccaaaggcuacaacuuguuucuuguugcugcgcaugaguucggccacuccuuaggucuugaccacuccaaggacccuggagcacucauguuuccuaucuacaccuacaccggcaaaagccacuuuaugcuuccugaugacgauguacaagggauccagucucucuaugguccaggagaugaagaccccaacccuaaacauccaaaaacgccagacaaaugugacccuuccuuaucccuugaugccauuaccagucuccgaggagaaacaaugaucuuuaaagacagauucuucuggcgccugcauccucagcagguugaugcggagcuguuuuuaacgaaaucauuuuggccagaacuucccaaccguauugaugcugcauaugagcacccuucucaugaccucaucuucaucuucagagguagaaaauuuugggcucuuaaugguuaugacauucuggaagguuaucccaaaaaaauaucugaacugggucuuccaaaagaaguuaagaagauaagugcagcuguucacuuugaggauacaggcaagacucuccuguucucaggaaaccaggucuggagauaugaugauacuaaccauauuauggauaaagacuauccgagacuaauagaagaagacuucccaggaauuggugauaaaguagaugcugucuaugagaaaaaugguuauaucuauuuuuucaacggacccauacaguuugaauacagcaucuggaguaaccguauuguucgcgucaugccagcaaauuccauuuugugguguuaagugucuuuuuaaaaauuguuauuuaaauccugaagagcauuugggguaauacuuccagaagugcgggguaggggaagaagagcuaucaggagaaagcuugguucugugaacaagcuucaguaaguuaucuuugaauauguaguaucuauaugacuaugcguggcuggaaccacauugaagaauguuagaguaaugaaauggaggaucucuaaagagcaucugauucuuguugcuguacaaaagcaaugguugaugauacuucccacaccacaaaugggacacauggucugucaaugagagcauaauuuaaaaauauauuuauaaggaaauuuuacaagggcauaaaguaaauacaugcauauaaugaauaaaucauucuuacuaaaaaguauaaaauaguaugaaaauggaaauuugggagagccauacauaaaagaaauaaaccaaaggaaaaugucuguaauaauagacuguaacuuccaaauaaauaauuuucauuuugcacugaggauauucagauguaugugcccuucuucacacagacacuaacgaaauaucaaagucauuaaagacaggagacaaaagagcagugguaagaauaguagauguggccuuugaauucuguuuaauuuucacuuuuggcaaugacucaaagucugcucucauauaagacaaauauuccuuugcauauuauaaaggauaaagaaggaugaugucuuuuuauuaaaauauuucagguucuucagaagucacacauuacaaaguuaaaauuguuaucaaaauagucuaaggccauggcaucccuuuuucauaaauuauuugauuauuuaagacuaaaaguugcauuuuaacccuauuuuaccuagcuaauuauuuaauuguccgguuugucuuggauauauaggcuauuuucuaaagacuuguauagcaugaaauaaaauauaucuuauaaaguggaaguauguauauuaaaaaagagacauccaaauuuuuuuuuaaagcagucuacuagauugugaucccuugagauauggaaggaugccuuuuuuucucugcauuuaaaaaaaucccccagcacuucccacagugccuauugauacuuggggagggugcuuggcacuuauugaauauaugaucggccaucaagggaagaacuauugugcucagagacacuguugauaaaaacucaggcaaagaaaaugaaaugcauauuugcaaaguguauuaggaaguguuuauguuguuuauaauaaaaauauauuuucaacagaaaaaaaa

SEQ ID NO: 2 - RNA, 39 nt, Homo sapiens
guggucaacugaugaguccgugaggucgaaaccuaagga

SEQ ID NO: 3 - RNA, 39 nt, Homo sapiens
caccaguucugaugaguccgugaggacgaauggauuccu

SEQ ID NO: 4 - RNA, 9 nt, Homo sapiens
gugguccaa

SEQ ID NO: 5 - RNA, 9 nt, Homo sapiens
accuaagga

SEQ ID NO: 6 - RNA, 7 nt, Homo sapiens
cugauga

SEQ ID NO: 7 - RNA, 4 nt, Homo sapiens
aaag

SEQ ID NO: 8 - DNA, 51 bp, Homo sapiens
tctagatccttaggtttcgtcctcacggactcatcagttgaccacgaattc

SEQ ID NO: 9 - DNA, 51 bp, Homo sapiens
gaattcgtggtcaactgatgagtccgtgaggacgaaacctaaggatctaga

SEQ ID NO: 10 - DNA, 50 bp, Homo sapiens
tctagaggaatccattcgtcctcacggactcatcagaactggtggaattc

SEQ ID NO: 11 - DNA, 50 bp, Homo sapiens
gaattccaccagttctgatgagtccgtgaggacgaatggattcctctaga

SEQ ID NO: 12 - DNA, 19 bp, Artificial Sequence
  Oligonucleotide primer
catccacgctgttttgacc

SEQ ID NO: 13 - DNA, 19 bp, Artificial Sequence
  Olignonucleotide primer
gaaatttgtgatgctattg

SEQ ID NO: 14 - DNA, 24 bp, Artificial Sequence
  Olignonucleotide primer
catttgatgggccctctggcctgc

SEQ ID NO: 15 - DNA, 24 bp, Artificial Sequence
  Olignonucleotide primer
gtttagggttggggtcttcatctc

SEQ ID NO: 16 - DNA, 21 bp, Artificial Sequence
  Olignonucleotide primer
cccatggcaaattccatggca

SEQ ID NO: 17 - DNA, 20 bp, Artificial Sequence
  Olignonucleotide primer
tctagacggcaggtcaggtc

SEQ ID NO: 18 - DNA, 49 bp, Homo sapiens
gaattcagggcccctgatgagtccgtgaggacgaaacaatgagtctaga

SEQ ID NO: 19 - DNA, 49 bp, Homo sapiens
tctagactcattgtttcgtcctcacggactcatcaggggccctgaattc

SEQ ID NO: 20 - DNA, 48 bp, Homo sapiens
gaattcattttgctgatgagtccgtgaggacgaaaccatttatctaga

SEQ ID NO: 21 - DNA, 48 bp, Homo sapiens
tctagataaatggtttcgtcctcacggactcatcagcaaaatgaattc

SEQ ID NO: 22 - DNA, 50 bp, Homo sapiens
gaattcgccttttcctgatgagtccgtgaggacgaaacttcagatctaga

SEQ ID NO: 23 - DNA, 50 bp, Homo sapiens
tctagatctgaagtttcgtcctcacggactcatcaggaaaaggcgaattc

(19) United States
(12) Patent Application Publication
(10) Pub. No.: US 2003/0195164 A1
(43) Pub. Date: Oct. 16, 2003

(54) Novel ribozyme and its use

(76) Inventors: Veli-Matti Kahari, Turku (FI); Risto Ala-Aho, Turku (FI); Matti Ahonen, Espoo (FI)

Correspondence Address: ROTHWELL, FIGG, ERNST & MANBECK, P.C., 1425 K STREET, N.W., SUITE 800, WASHINGTON, DC, 20005, US

(21) Appl. No.: 10/372,232

(22) Filed: Feb. 25, 2003

Related U.S. Application Data
(60) Provisional application No. 60/372,422, filed on Apr. 16, 2002.

(51) Int. Cl. A61K 48/00; A61K 31/724; C07H 21/02; C12N 15/85; C12Q 1/68; C12P 19/34

(52) U.S. Cl. 514/44; 514/58; 536/23.1; 435/320.1; 435/455; 435/6; 435/91.2

(57) ABSTRACT

This invention concerns an enzymatic RNA molecule which is capable of specifically cleaving matrix metalloproteinase 13 (MMP-13) (also called collagenase-3) messenger RNA. The invention concerns further a pharmaceutical composition comprising the novel ribozyme and an expression vector encoding the same, and a composition comprising said vector. Furthermore, the invention concerns further a method for reducing or eliminating the expression of MMP-13 in vivo; a method for treating or preventing cancer, or preventing or inhibiting cancer growth, invasion or metastasis; and a method for treating or preventing various inflammatory conditions. The invention concerns also methods for detecting the level of MMP-13 in a tissue or body fluid, and the use of this information for the diagnosis of MMP-13 related diseases.

### FIELD OF THE INVENTION

This invention concerns a novel ribozyme, a pharmaceutical composition comprising the same and an expression vector encoding the same, and a composition comprising said vector. The invention concerns further a method for reducing or eliminating the expression of matrix metalloproteinase 13 (MMP-13), also called collagenase-3, in vivo. Furthermore, the invention concerns a method for treating or preventing cancer, or preventing or inhibiting cancer growth, invasion or metastasis; or a method for treating or preventing inflammatory conditions, especially osteoarthritis, rheumatoid arthritis, rupture of atherosclerotic plaque, aorta aneurysm, congestive hearth failure, chronic skin wounds, gastrointestinal ulcer, or chronic periodontitis or gingivitis in a person. Still further, the invention concerns a method for detecting or quantifying the level of MMP-13 in a tissue or fluid and the use of such information for diagnosing an MMP-13 related cancer or MMP-13 related inflammatory conditions in an individual.

### BACKGROUND OF THE INVENTION

The publications and other materials used herein to illuminate the background of the invention, and in particular, cases to provide additional details respecting the practice, are incorporated by reference.

Tumor invasion and metastasis involves detachment of cancer cells from primary tumor, controlled degradation of structural barriers, such as basement membrane and collagenous extracellular matrix (ECM), and migration of cells through degraded matrix. Matrix metalloproteinases (MMPs) are a family of zinc-dependent neutral endopeptidases collectively capable of degrading essentially all ECM components and they obviously play an important role in tumor invasion and tumor-induced angiogenesis (Westermarck and Kähäri 1999). At present, 21 human members of the MMP gene family are known and they are divided into subgroups of collagenases, gelatinases, stromelysins, membrane-type MMPs, and other MMPs according to their structure and substrate specificity (Johansson et al. 2000). In addition to the ECM substrates, MMPs also cleave cell surface molecules and other pericellular non-matrix proteins, such as growth factors, cytokines, chemokines and their receptors, and activate other proteinases thereby regulating cell behaviour in several ways.

Fibrillar collagens are the most abundant structural components of the human connective tissues and it is conceivable, that the ability to degrade them is crucial for invasion and metastasis of neoplastic cells. Members of the collagenase subgroup, i.e. collagenase-1 (MMP-1), collagenase-2 (MMP-8), and collagenase-3 (MMP-13) are the only neutral proteinases capable of cleaving native fibrillar collagens of type I, II, III, and V (Kähäri and Saarialho-Kere 1997). MMP-13 also degrades several other ECM components: type IV, X, and XIV collagens, large tenascin C, fibronectin, aggrecan, versican, and fibrillin-1 (Ashworth et al. 1999; Fosang et al. 1996; Knäuper et al. 1997; Knäuper et al. 1996). In normal tissues, the expression of MMP-13 is limited to physiologic situations, in which rapid and effective remodeling of collagenous ECM is required, i.e. fetal bone development (Johansson et al. 1997b) and gingival wound repair (Ravanti et al. 1999b). The wide proteolytic substrate specificity of MMP-13 suggests a role for it as a powerful invasion tool for malignant cells, and in fact, expression of MMP-13 has been detected in various invasive neoplastic tumors, i.e. breast carcinomas (Heppner et al. 1996), squamous cell carcinomas (SCCs) of the head and neck (Airola et al. 1997; Cazorla et al. 1998; Johansson et al. 1997a), vulva (Johansson et al. 1999), and esophagus (Etoh et al. 2000), in chondrosarcomas (Uria et al. 1998), primary and metastatic melanomas (Airola et al. 1999; Nikkola et al. 2001), and urothelial carcinomas (Boström et al. 2000). In SCCs of the skin, oral cavity, pharynx, larynx, and vulva MMP-13 is expressed primarily by cancer cells at the invading edge of the tumor and its expression correlates with the invasion capacity of the tumors (Airola et al. 1997; Cazorla et al. 1998; Etoh et al. 2000; Johansson et al. 1997a; Johansson et al. 1999). However, no expression of MMP-13 is noted in premalignant tumors in human skin, or normal epidermal keratinocytes in culture or in vivo (Johansson et al. 1997c; Vaalamo et al. 1997). These observations show, that MMP-13 expression serves as a marker for transformation of squamous epithelial cells and suggest a role for MMP-13 in invasion of SCC cells at an early stage of tumor growth.

In addition to invasive carcinomas, expression of MMP-13 is detected in some other pathologic conditions characterized by destruction of normal collagenous tissue architecture in osteoarthritic cartilage, rheumatoid synovium, chronic cutaneous ulcers, intestinal ulcerations, chronic periodontitis, atherosclerosis, and aortic aneurysms (Lindy et al. 1997; Mao et al. 1999; Reboul et al. 1996; Sukhova et al. 1999; Uitto et al. 1998; Vaalamo et al. 1998; Vaalamo et al. 1997).

Antisense oligonucleotides and catalytic RNAs such as hammerhead ribozymes are capable of modulating specific gene expression and they have demonstrated utility in attenuating eukaryotic gene expression (Scanlon et al. 1995). Compared to traditional antisense techniques, ribozymes are site specific and their catalytic potential makes them more efficient in suppressing the specific gene expression. Ribozymes have been developed as novel therapeutic agents that can suppress deleterious proteins by catalyzing the trans-cleavage of the corresponding mRNAs (Santiago and Khachigian 2001). Small-molecular agents acting as MMP-13 inhibitors for treatment of MMP-13 related diseases have been disclosed in the art.

Because the MMP-13 mRNA is not expressed in most normal adult human tissues, down-regulating MMP-13 expression may be an important strategy for specific gene therapy of cancer and other MMP-13 related diseases.

### SUMMARY OF THE INVENTION

A basis for the present invention is the discovery that there exists correlation between expression of MMP-13 and cancer invasion, cancer growth and inflammatory conditions in certain tissues and that the level of MMP-13 can be suppressed in a novel manner. The study referred in detail in the Experimental Section shows that suppression of the MMP-13 expression results in suppressed cancer invasion, reduced cancer cell proliferation, reduced cancer growth and increased cancer cell apoptosis.

This invention offers an effective method of reducing or eliminating the expression of MMP-13, namely by use of a novel ribozyme specifically cleaving the MMP-13 mRNA.

Thus, in its broadest aspect, this invention concerns an enzymatic RNA molecule (or ribozyme) which is capable of specifically cleaving a target RNA molecule, which is MMP-13 messenger RNA.

According to another aspect, the invention concerns a pharmaceutical composition comprising a therapeutically effective amount of the enzymatic RNA molecule, either in its unmodified or modified form, in a pharmaceutically acceptable carrier.

According to a third aspect, the invention concerns an isolated mammalian cell, especially a human cell, including the enzymatic RNA molecule, either in its unmodified or modified form.

According to a fourth aspect, the invention concerns an expression vector including nucleic acid encoding the enzymatic RNA molecule according to this invention, in a manner which allows expression of said enzymatic RNA within a mammalian cell as well as a pharmaceutical preparation comprising said vector.

According to a fifth aspect, the invention concerns a method for reducing or eliminating the expression of MMP-13 in an individual, said method comprising administering to said individual

i) an effective amount of the enzymatic RNA, either in its unmodified or modified form, or

ii) an expression vector including nucleic acid encoding the enzymatic RNA molecule, in a manner which allows expression of said enzymatic RNA within a mammalian cell.

According to a sixth aspect, the invention concerns a method for treating or preventing cancer, or preventing or inhibiting cancer growth, invasion or metastasis in an individual, said method comprising administering to said individual

i) an effective amount of the enzymatic RNA, either in its unmodified or modified form, or

ii) an expression vector including nucleic acid encoding the enzymatic RNA molecule according to this invention, in a manner which allows expression of said enzymatic RNA within a mammalian cell.

According to a seventh aspect, this invention concerns a method for inducing of cancer cell apoptosis in an individual, comprising inhibiting expression or inhibiting or suppressing the activity of MMP-13 in said individual.

According to an eighth aspect, the invention concerns a method for treating or preventing an inflammatory condition, especially osteoarthritis, rheumatoid arthritis, rupture of atherosclerotic plaque, aorta aneurysm, congestive hearth failure, chronic skin wounds, gastrointestinal ulcer, or chronic periodontitis or gingivitis in an individual, said method comprising administering to said individual

i) an effective amount of the enzymatic RNA molecule, either in its unmodified or modified form, or

ii) an expression vector including nucleic acid encoding the enzymatic RNA according to this invention, in a manner which allows expression of said enzymatic RNA within a mammalian cell.

According to a ninth aspect, this invention concerns a method for detecting or quantifying the level of MMP-13 in a tissue or body fluid by

i) determining the MMP-13 mRNA expression from said tissue or fluid by RT-PCR or by a hybridizing technique, or

ii) subjecting the tissue or body fluid expected to contain the protein MMP-13 to an antibody recognizing MMP-13, and detecting and/or quantifying said antibody, or subjecting said tissue or body fluid to analysis by proteomics technique.

### BRIEF DESCRIPTION OF THE DRAWINGS

FIG. 1. The structure of MMP-13 ribozyme and in vitro cleavage of MMP-13 mRNA by antisense ribozyme. A. The MMP-13 antisense ribozyme targets human MMP-13 mRNA between nucleotides +707 and +724. The predicted cleavage site is between nucleotides +716 and +717. The flanking vector-generated sequences are not shown. Control sense hammerhead ribozyme contains catalytic loop of hammerhead ribozyme but has no sequence complementary to MMP-13 mRNA. B. Expected cleavage fragments of MMP-13 transcript with MMP-13 antisense ribozyme. C. In vitro cleavage of human MMP-13 mRNA by ribozyme. MMP-13 mRNA was incubated with antisense ribozyme for different periods of time (0 to 8 hrs) or with sense ribozyme for 8 hours and analyzed by electrophoresis on agarose gel and visualized by ethidium bromide. The size of uncleaved MMP-13 mRNA and specific cleavage fragments are indicated at left.

FIG. 2. Adenoviral expression of MMP-13 antisense ribozyme inhibits MMP-13 expression and invasion of squamous carcinoma cells. Human cutaneous squamous carcinoma (SCC) cells (UT-SCC-7) (A) and ras-transformed HaCaT keratinocytes (B) were infected with recombinant adenoviruses RAdMMP-13ASRz harboring human MMP-13 antisense hammerhead ribozyme sequence and RAdMMP-13 senseRz harboring MMP-13 sense ribozyme sequence at appropriate MOI for 6 h. Production of MMP-13 and MMP-1 was determined by Western blot analysis and the levels of 92 kDa and 72 kDa gelatinases were analyzed by gelatin zymography of the conditioned media at different time points after infection, as indicated. C. Cell culture inserts were pre-coated with 25 μg Matrigel. UT-SCC-7 cells were infected with RAdMMP-13ASRz or RAdMMP-13senseRz for 6 h and seeded on top of Matrigel. The number of invaded cells were determined after 24 h. Mean+SEM of 2 experiments performed in duplicate are shown. Statistical significance against uninfected control cells was determined by Student's t test: * p<0.05.

FIG. 3. MMP-13 antisense ribozyme suppresses the growth of squamous carcinoma cells in vitro and induces apoptosis. A. UT-SCC-7 cells (right panel) and HaCaT cells (left panel) were infected with RAdMMP-13ASRz and RAdMMP-13 sense and the number of cells was determined at different time-points by MTT assay. The mean+SD are shown (n=4). *p<0.002 by Student's t-test. B. 20 000 UT-SCC-7 cells were seeded onto plates and infected with recombinant adenoviruses as above and the number of cells were counted at different time points. The results represent mean±SD of three plates. *Antisense vs.PBS or pCA3, p<0.002; antisense vs. sense p<0.05. C. The cultured UT-SCC-7 cells were infected as above and fragmented DNA was stained with TUNEL reaction three days after infections. Nuclei of the SCC cells were stained three and four days after infection with Hoechst 33342 to show chromatin structure.

FIG. 4. Adenovirus mediated delivery of MMP-13 antisense ribozyme inhibits tumor growth in vivo. A. UT-SCC-7 cells in culture were infected with recombinant adenoviruses expressing MMP-13 antisense ribozyme (RAdMMP-13ASRz) or MMP-13 sense control ribozyme (RAdMMP-13senseRz) at MOI 700 for 6 hours. On the following day, cells (5×10⁶) were implanted subcutaneously in the back of SCID/SCID mice and the size of tumors was measured once a week. Statistical significance between RAdMMP-13ASRz infected and RAdMMP-13senseRz or PBS injected groups were determined by Student's t-test: * p<0.01. B. Subcutaneous SCC tumors were established by injecting 5×10⁶ UT-SCC cells in the back of SCID mice. The tumors were injected with RAdMMP-13ASRz and RAdMMP-13senseRz.(1×10⁹ pfu) twice a week starting on day 41 and the size of tumors was measured at the time of injection. Statistical significance between RAdMMP-13ASRz infected and RAdMMP-13senseRz of PBS injected groups was determined by Student's t-test: * p<0.05. C. Subcutaneous SCC tumors were established as in B and were injected three times a week starting on day 36 and the size of tumors was measured at the time of injection.. Statistical significance between RAdMMP-13ASRz and RAdMMP-13senseRz treated groups: * p<0.05, ** p<0.01.

FIG. 5. Adenoviral expression of MMP-13 antisense ribozyme inhibits MMP-13 expression and gelatinolytic activity in squamous cell carcinomas. Subcutaneous SCC tumors were established by injecting 5×10⁶ UT-SCC cells in the back of SCID mice. The tumors were injected with recombinant adenoviruses expressing MMP-13 antisense ribozyme (RAdMMP-13ASRz) or MMP-13 sense control ribozyme (RAdMMP-13senseRz) (1×10⁹ pfu) three times a week starting on day 36 (FIG. 4C) and analyzed 20 days later. A. Total RNA was isolated from tumor tissue and RT-PCR was done to study expression level of MMP-13 mRNA in adenoviral injected tumors. B. Gelatinolytic activity in tumors determined with in situ gelatinase zymography. Gelatinase acitivity is noted as white areas of gelatin degradation in PBS and RAdMMP-13senseRz injected tumors (upper panel). The hematoxylin and eosin staining of the same tissue sections are shown underneath.

FIG. 6. RAdMMP-13ASRz suppresses proliferation of tumor cells SCC tumors in SCID mice. Subcutaneous SCC tumors were established by injecting 5×10⁶ UT-SCC cells in the back of SCID mice. The tumors were injected with recombinant adenoviruses expressing MMP-13 antisense ribozyme (RAdMMP-13ASRz) or MMP-13 sense control ribozyme (RAdMMP-13senseRz) (1×10⁹ pfu) three times a week starting on day 36 (FIG. 4C) and analyzed 20 days later. A. SCC tumors were immunostained for Ki67 as a marker of proliferating cells. B. The Ki67 positive area was measured and compared to average tumor sizes.

FIG. 7 shows the human MMP-13 mRNA, the start and stop codons between which the MMP-13 protein coding region exists, and preferable sites to be cleaved by a hammerhead ribozyme according to this invention.

FIG. 8 shows the human MMP-13 mRNA according to FIG. 7 and the preferable sites to be cleaved by a hairpin ribozyme according to this invention.

### DETAILED DESCRIPTION OF THE INVENTION

The Ribozyme

The “enzymatic RNA molecule” or ribozyme shall be understood as a nucleotide sequence comprising exclusively ribonucleotides, or a sequence comprising of ribonucleotides and 2′-deoxyribonucleotides. The latter sugar units may, as will be disclosed later, be useful for stabilizing the ribozyme.

The wording “specifically cleaving” means that the ribozyme according to this invention does not cleave other RNA:s than the target mRNA as defined herein.

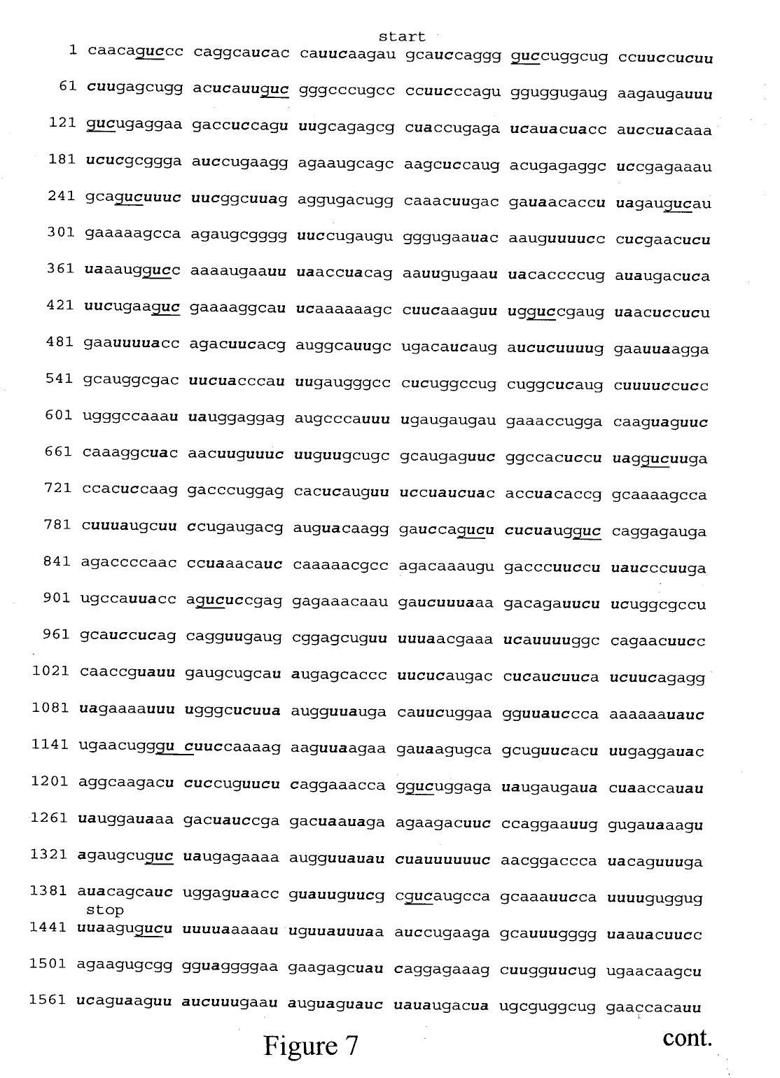

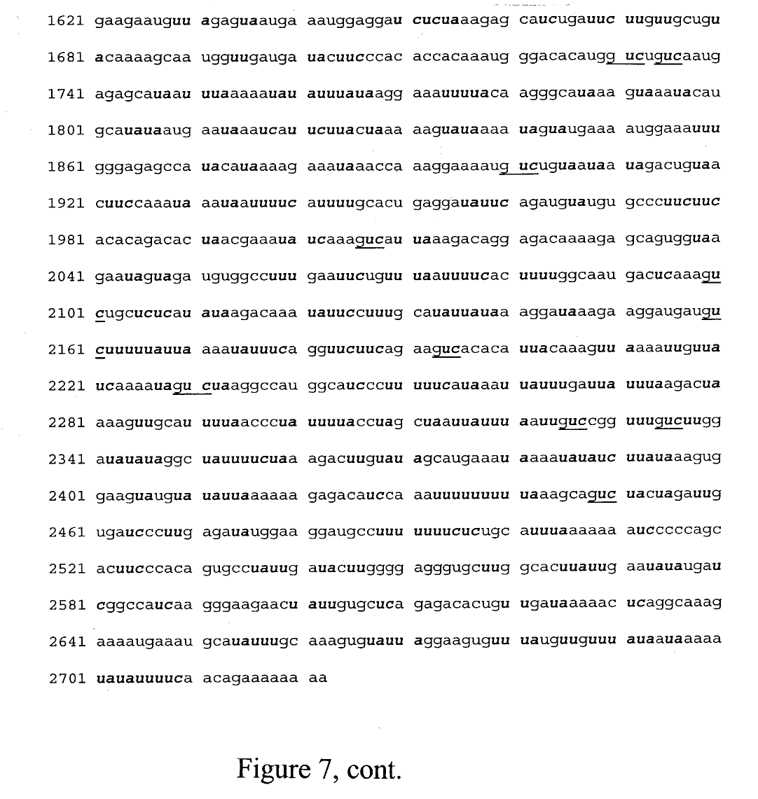

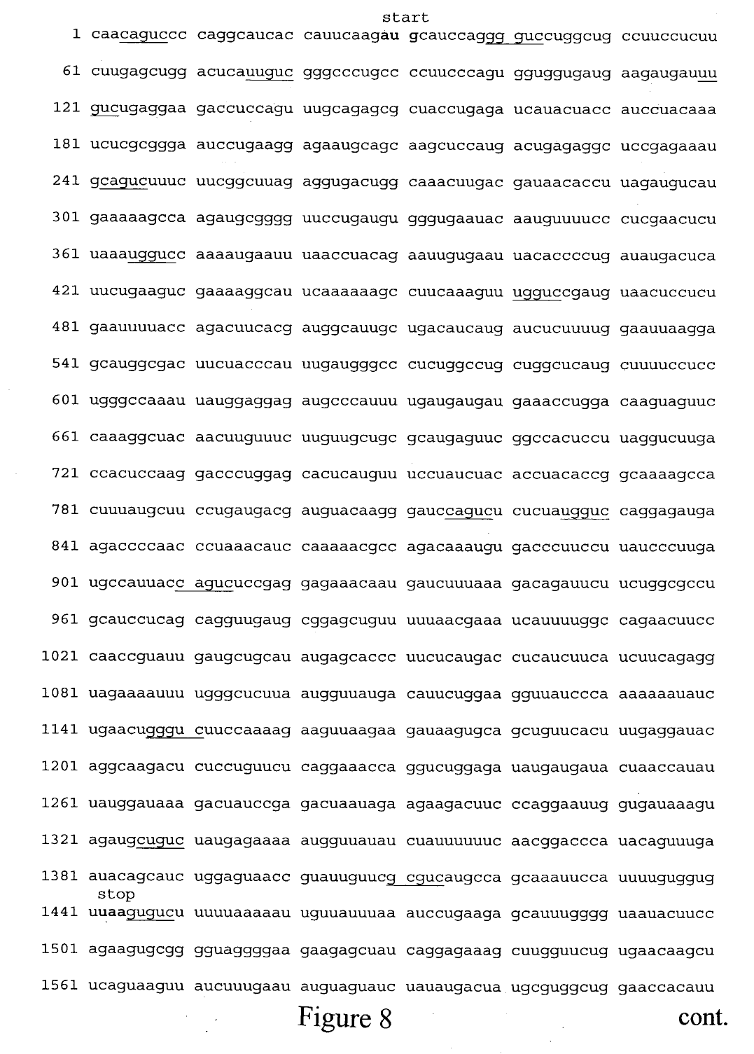

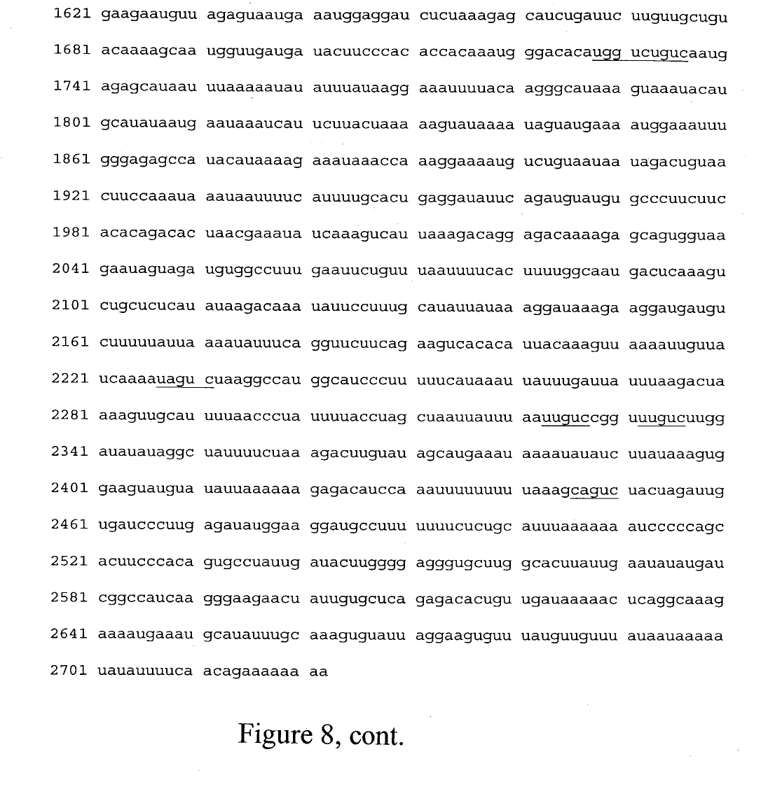

The human MMP-13 mRNA is a ribonucleotide sequence obtainable from GenBank and is shown in FIGS. 7 and 8. The start and stop codons between which the MMP-13 protein coding region exists are indicated (start nt 29 and stop nt 1444).

The ribozyme according to this invention can comprise a hammerhead motif, a hairpin motif, a hepatitis delta virus motif, RNaseP RNA or Neurospora VS RNA. The hammerhead or hairpin motifs are preferable, especially the hammerhead motif.

A typical feature of the hammerhead ribozyme according to this invention is that it can catalytically cleave the target RNA, i.e. MMP-13 mRNA, after any sequence UH in the target RNA, where U is a uridine nucleotide and H is an adenosine nucleotide, a cytidine nucleotide or a uridine nucleotide. Thus, H can contain any base except for guanosine. These sequences are indicated by bold italic letters in FIG. 7.

More preferably, the hammerhead ribozyme according to this invention is capable of specifically cleaving the target RNA after any GUC-sequence in the target RNA. Such cleavage sites appear in the target RNA sequence at the underlined positions in FIG. 7.

In case the ribozyme according to this invention comprises a hairpin motif, it is preferably capable of specifically cleaving the target RNA after any sequence BNGUC in the target RNA, where B is a cytosine nucleotide, a guanosine nucleotide or a uridine nucleotide; N is any nucleotide and G is a guanosine nucleotide, U is a uridine nucleotide and C is a cytidine nucleotide. Such cleavage sites appear in the target RNA sequence at the underlined positions in FIG. 8.

The wording expressing that the cleavage site is located “after” a certain sequence means that the cleaving site is on the 3′-side of the sequence in question.

The cleavage site is preferably located within the MMP-13 protein coding region of the MMP-13 mRNA, i.e. between the start and stop codons.

According to a preferred embodiment, the ribozyme according to this invention comprises two nucleotide sequences complementary to two nucleotide sequences of the target RNA, each located on different sides of the cleavage site in the target RNA, and a catalytic cleaving sequence.

The term “complementary” means that the nucleotide sequence can form hydrogen bonds with the target RNA sequence by Watson-Crick or other base-pair interactions. The term shall be understood to cover also sequences which are not 100% complementary. It is believed that lower complementarity, even as low as 50% or more, may work. However, 100% complementarity is preferred.

According to a preferred embodiment, the ribozyme comprises a hammerhead motif. The catalytic cleaving sequence consists preferably of two different ribonucleotide sequences (a first catalytic ribonucleotide sequence and a second catalytic ribonucleotide sequence) wherein the catalytic ribonucleotide sequences are bound to separate complementary nucleotide sequences. The other ends of the catalytic sequences are bound to a nucleotide sequence capable of base pairing inter se.

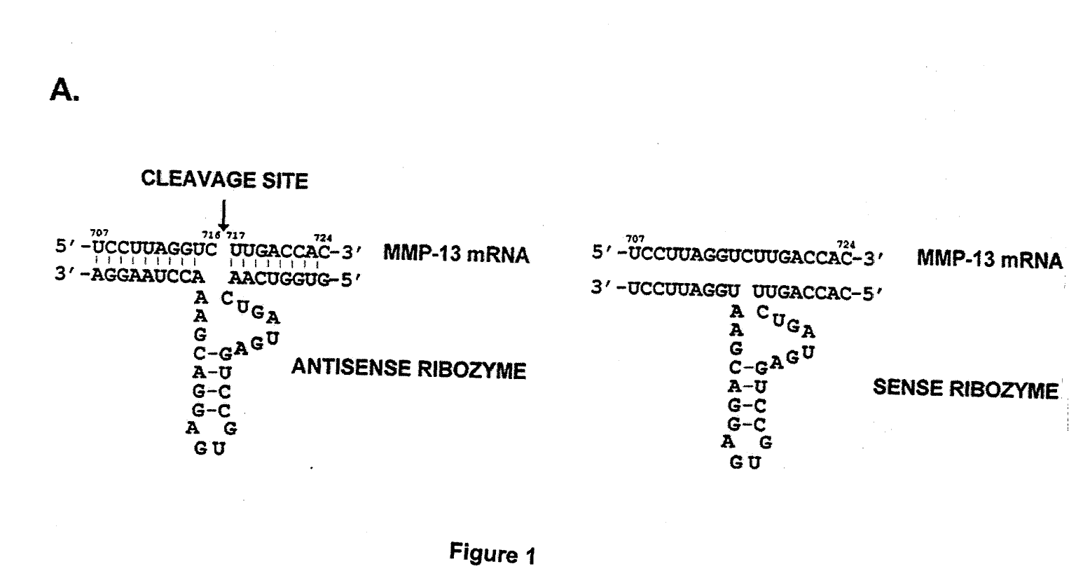

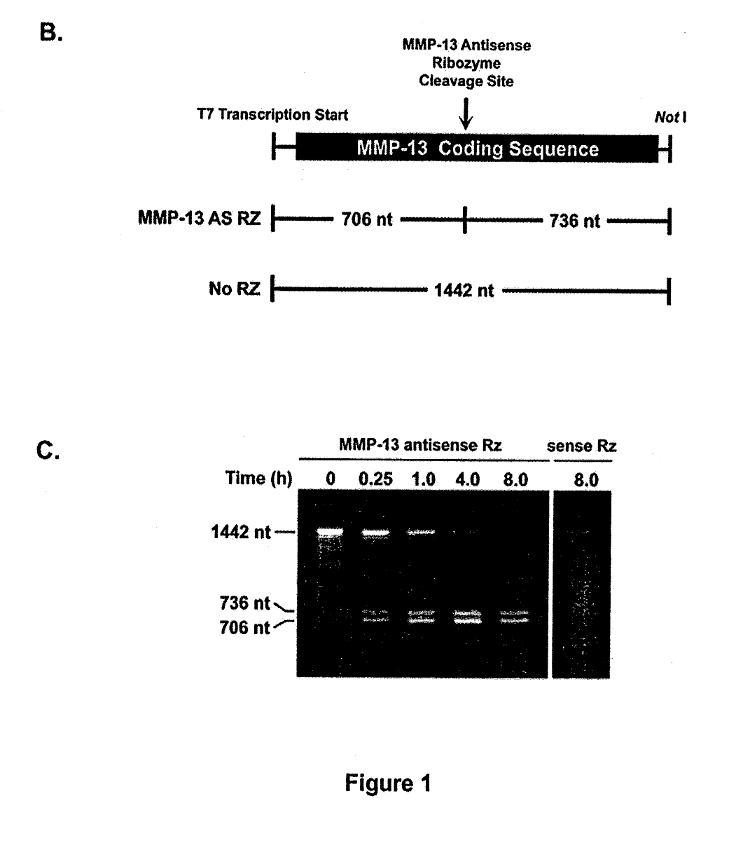

More preferably, the ribozyme has a first complementary nucleotide sequence which is 5′-GUGGUCAA-3′ and a second complementary nucleotide sequence which is 5′-ACCUAAGGA-3′. The catalytic cleaving sequence forms a first catalytic ribonucleotide sequence CUGAUGA and a second catalytic ribonucleotide sequence AAAG. These catalytic ribonucleotide sequences are bound to a separate complementary nucleotide sequence and to a nucleotide sequence capable of base pairing inter se. This ribozyme is capable of cleaving human MMP-13 mRNA between the nucleotides 716 and 717 as shown in FIG. 1A and 7.

Other preferable cleaving sites are between the nucleotides 80-81; 369-370; and 430-431. These ribozymes are shown in the experimental section.

The ribozyme should preferably not be longer than 60 nucleotides, more preferably not longer than 50 nucleotides. The synthesis and administration of the ribozyme molecules is easier if the sequence is not very long.

An especially preferable ribozyme is the antisense ribozyme shown in FIG. 1 A.

To construct an alternative ribozyme, designed to cleave the target RNA after another sequence in the target RNA than that disclosed in FIG. 1 A, it is of course necessary to create appropriate antisense sequences so that such a ribozyme will be capable to hybridize to the target RNA sequence in the proximity to the selected cleavage site.

Although antisense sequences comprising only 5 nucleotides per chain might work, it is believed that a preferable length is 6 to 7 nucleotides per chain, or more preferably 8 to 9 nucleotides per chain.

Modifications of the Ribozyme

The ribozyme shall, when used as a pharmaceutical, be introduced in a target cell. The delivery can be accomplished, as will be dealt with in more detail in the following section, in two principally different ways: 1) exogenous delivery of the ribozyme, or 2) endogenous transcription of a DNA sequence encoding this ribozyme, where the DNA sequence is located in a vector.

Normal, unmodified RNA has low stability under physiological conditions because of its degradation by ribonuclease enzymes present in the living cell. If the ribozyme shall be administered exogenously, it is highly desirable to modify the ribozyme according to known methods so as to enhance its stability against chemical and enzymatic degradation.

Modifications of ribozymes are extensively disclosed in prior art. Reference is made to Draper et al., U.S. Pat. No. 5,612,215, which in turn lists a number of patents and scientific papers concerning this technique. It is known that removal of the 2′-OH group from the ribose unit gives a better stability, but may lead to a reduced cleaving activity of the ribozyme. Rossi et al., WO 91/03162 discloses a hammerhead ribozyme cleaving mRNA of HIV-1. In this ribozyme, ribonucleotides in the antisense chains and in the chain base-pairing inter se were replaced by 2′deoxyribonucleotides, but no changes were made in the cleaving sequences. Eckstein et al., WO 92/07065 and U.S. Pat. No. 5,672,695 discloses the replacement of the ribose 2′-OH group with halo, amino, azido or sulfhydryl groups. Sproat et al., U.S. Pat. No. 5,334,711, discloses the replacement of hydrogen in the 2′-OH group by alkyl or alkenyl, preferably methyl or allyl groups. Furthermore, the internucleotidic phosphodiester linkage can, for example, be modified so that one ore more oxygen is replaced by sulfur, amino, alkyl or alkoxy groups. Also the base in the nucleotides can be modified. The ribose units and the internucleotidic linkages can be modified to a great extent in the antisense chains, while only very few, preferably only one of the ribose units in the cleaving sequence should be modified. Usman el al., U.S. Pat. No. 5,652,094 and Jennings et al., WO 94/13688 describe further modified ribozymes. Draper et al., U.S. Pat. No. 5,612,215 suggests a modified stromelysin mRNA cleaving ribozyme in a hammerhead motif where the 2′-OH groups in the antisense chains are replaced by 2′-O-methyl and the internucleotide linkages in the antisense chains are phosphorothioate linkages. Furthermore, in one of the ribonucleotides in the cleaving region, 2′-OH was replaced by 2′-O-allyl groups. Usman and Blatt, 2000, disclose a new class of nuclease-resistant ribozymes, where the 3′ end can be protected by the addition of an inverted 3′-3′ deoxyabasic sugar.

Preferable modifications are, for example the RNA molecule wherein one or more of the 2′-OH groups in the complementary nucleotide sequences are replaced by 2′-O-methyl. Even more preferable is an RNA molecule where a 2′-OH group in the catalytic cleaving nucleotide sequence is replaced by 2′-O-allyl, the internucleotide phoshodiester linkage in the complementary sequences are modified, e.g. replaced by phosphorothioate linkages and the 3′ end of the RNA molecule is protected by the addition of an inverted 3′-3′ deoxyabasic sugar.

Especially preferable is the RNA molecule, wherein some or all of the ribonucleotides in the complementary chains have modifications in the 2′-OH groups of their ribose units and/or modifications in their internucleotidic phosphodiester linkages and/or the RNA molecule has an inverted 3′-3′-deoxyabasic sugar added to its 3′-end, and the 2′-OH group in the ribose unit of at least one of the ribonucleotides in the catalytic cleaving sequence is modified, for example by replacement with a 2′-O-allyl group.

The unmodified as well as the modified ribozymes can be prepared according to the methods disclosed in the cited patent publications and other prior art publications.

Administration of the Ribozyme

The ribozymes according to this invention can be administered to the individual by various methods. According to one method, the ribozyme may be administered as such, as complexed with a cationic lipid, packed in a liposome, incorporated in cyclodextrins, bioresorbable polymers or other suitable carrier for slow release administration, biodegradable nanoparticle or a hydrogel. For some indications, ribozymes may be directly delivered ex vivo to cells or tissues with or without the aforementioned vehicles.

The ribozyme can be administered systemically or locally. As suitable routes of administration can be mentioned intravenous, intramuscular, subcutaneous injection, inhalation, oral, topical, systemic, ocular, sublingual, nasal, rectal, intraperitoneal delivery and iontophoresis or other transdermal devivery systems. The composition containing the RNA can, instead of using direct injection, also be administered by use of, for example, a catheter, infusion pump or stent. Furthermore, the ribozyme or the composition containing the same can be included in a coating on an endo-osteal prosthesis or a dental implant.

According to one embodiment, the pharmaceutical composition containing the novel ribozyme is an oral hygiene product such as a toothpaste or a mouthwash or any other product aimed to target the dental tissue in order to facilitate treatment or prevention of chronic periodontitis or gingivitis.

Another method to achieve high concentrations of the ribozyme in cells, is to incorporate the ribozyme-encoding sequence into an expression vector and to administer such a vector to the individual. The expression vector can be a DNA sequence, such as a DNA plasmid capable of eukaryotic expression, or a viral vector. Such a viral vector is preferably based on an adenovirus, an alphavirus, an adeno-associated virus, a retrovirus or a herpes virus. Preferably, the vector is delivered to the patient in similar manner as the ribozyme described above. The delivery of the expression vector can be systemic, such as intravenous, intramuscular or intraperitoneal administration, or local delivery to target tissue or to cells explanted from the patient, followed by reintroduction into the patient.

Use of the Ribozyme

As this invention offer a novel method for reducing or eliminating the expression of MMP-13 in an individual, any disease or disorder related to the appearance of MMP-13 can be treated or prevented by this method. Thus, this invention covers also treating or preventing other diseases than those explicitly mentioned here.

The treatment or prevention of cancer or prevention of cancer metastasis is, as will be shown in the Experimental Section, based on i) suppressing invasion of cancer cells, or ii) inhibiting tumor growth, or iii) inducing cancer cell apoptosis, or a combination of these mechanisms.

This method is especially suitable for treating or preventing of cancers located in certain tissues and cancers that would be difficult or impossible to treat by surgery or radiation. As examples of such cancers can be mentioned squamous cell carcinomas on the skin, in the oral cavity, pharynx or larynx, vulval cancers, primary and metastatic melanomas, urothelial carcinomas, and osteosarcomas, condrosarcoma, breast carcinoma, uterine cervix carcinoma and esophagus carcinomas.

The method according to this invention can be accomplished either as the sole treating or preventing method, or as an adjuvant therapy, combined with other methods such as administration of cytotoxic agents, surgery, radiotherapy, immunotherapy etc..

As examples of inflammatory conditions that can be treated or prevented can be mentioned osteoarthritis, rheumatoid arthritis, rupture of atherosclerotic plaque, aorta aneurysm, congestive hearth failure, chronic skin wounds, gastrointestinal ulcer, and chronic periodontitis or gingivitis.

So far, it has been suggested to treat MMP-13 related diseases with small-molecular inhibitors. Very often, drugs in the form of small-molecular inhibitors are not specific enough for the target enzyme, and are thereby likely to induce untoward side-effects or adverse effects. It is expected that the method according to this invention offers a more selective way of treating or preventing such diseases because this protein is very selectively expressed in adult patients in disease related tissues.

This invention concerns further a method for detecting or quantifying the level of MMP-13 in a tissue or body fluid by either

i) determining the MMP-13 mRNA expression from said tissue or fluid by RT-PCR, or by a hybridizing technique, or

ii) subjecting the tissue or body fluid expected to contain the protein MMP-13 to an antibody recognizing MMP-13, and detecting and/or quantifying said antibody, or subjecting said tissue or body fluid to analysis by proteomics technique.

The hybridizing technique include, for example DNA hybridization and northern blot. The detection or quantification of the antibody can be performed according to standard immunoassay protocols, such as label-linked immunosorbent assays, western blot and immunohistochemical methods.

This method for detection or quantifying MMP-13 can be used in vitro to investigate the effect of novel ribozymes, expected to specifically cleave MMP-13 mRNA. Alternatively, the method can be used for diagnosing an MMP-13 related disease or condition, especially for diagnosing an MMP-13 related cancer or an MMP-13 related inflammatory condition, such as osteoarthritis, rheumatoid arthritis, rupture of atherosclerotic plaque, aorta aneurysm, congestive hearth failure, chronic skin wounds, gastrointestinal ulcer, or chronic periodontitis or gingivitis.

The dose of the ribozyme will depend on the disease to be treated or prevented, the modification of the ribozyme, on the carrier and on the administration route. The final dose shall be established by clinical trials. However, based on published ribozyme dosages in animal experiments (Pavco et al., 2000), it is believed that the suitable daily dose is between 1 and 100 mg per kg body weight.

Induction of Cancer Cell Apoptosis

The experiments disclosed below illustrate for the first time that cancer cell apoptosis can be induced by suppressing MMP-13. This can be performed by inhibiting the expression of MMP-13 or by inhibiting or suppressing the activity of MMP-13. The expression of MMP-13 can be inhibited by the ribozyme disclosed above, but also, for example, with an MMP-13 mRNA antisense oligonucleotide or a short interfering RNA. The activity of the MMP-13 protein can, for example, be inhibited or suppressed by treatment with a small molecule MMP-13 inhibitor or an intracellular or extracellular activity blocking antibody.

The invention will be illuminated more in detail by the following non-restrictive Experimental Section.

### EXPERIMENTAL SECTION

### Materials and Methods

Cell Cultures

Human SCC cell line UT-SCC-7, established from metastasis of cutaneous SCC (Servomaa et al., 1996) was cultured in DMEM supplemented with 6 mmol/L glutamine, nonessential amino acids, and 10% fetal calf serum (FCS). HaCaT cells and A-5 cells, a ras-transformed tumorigenic HaCaT cell line (Boukamp et al. 1990) was cultured in Dulbecco's modified Eagle's medium (DMEM) containing 10% FCS.

Design of MMP-13 Ribozyme

A MMP-13 antisense ribozyme was designed to target nucleotides 707-724 at the coding region of the human MMP-13 mRNA sequence with the cleavage site targeted between the nucleotides 716 and 717 (FIG. 1 A). Corresponding hammerhead control was designed in sense orientation to the same nucleotides. The following oligonucleotides were used for cloning MMP-13 ribozyme expression vectors. The flanking restriction enzyme cleavage sites are undelined.

| | |
|---|---|
| Oligonucleotide | Sequence |

| | |
|---|---|
| MMP-13 antisense Rz rev | 5′-TCTAGATCCTTAGGTTTCGTCCTCACGGACTCATCAGTTGACCACGAATTC-3′ |
| MMP-13 antisense Rz frw | 5′-GAATTCGTGGTCAACTGATGAGTCCGTGAGGACGAAACCTAAGGATCTAGA-3′ |
| MMP-13 senseRz rev | 5′-TCTAGAGGAATCCATTCGTCCTCACGGACTCATCAGAACTGGTGGAATTC-3′ |
| MMP-13 senseRz frw | 5′-GAATTCCACCAGTTCTGATGAGTCCGTGAGGACGAATGGATTCCTCTAGA-3′ |

Equal amounts of reverse (rev) and forward (frw) oligonucleotides were heated to 80° C. and allow to cool to room temperature and anneal to form MMP-13 antisense and MMP-13 sense ribozyme coding double-stranded DNA molecules.

The double stranded DNA molecules were subcloned into pCI-neo vector (Promega) digested with EcoRI and XbaI, and the correct orientation of the inserts was verified by nucleotide sequencing. Antisense and sense MMP-13 ribozymes were generated by in vitro transcription from pCI-neo-ribozyme vectors linearized with NotI using T7 RNA polymerase. MMP-13 mRNA was transcribed from pCI-MMP13neo plasmid (Ala-aho et al. 2002b) linearized with NotI resulting in RNA molecule of 1442 nucleotides in length (FIG. 1 B). Both ribozyme RNA and the target MMP-13 RNA were heated to 80° C. in the presence of 10× reaction buffer (500 mM Tris-HCl, pH 7.5, 10 mM EDTA and 500 mM NaCl), and allowed to cool to room temperature. DTT (at the final concentration 10 μM) RNase inhibitor (10 U) and MgCl₂ (20 mM) was added and the mixtures of ribozyme and target RNA was incubated at 37° C. different periods of time. Reactions were stopped by the addition of 5×RNA loading buffer. Reaction products were fractionated on a 5% polyacrylamide gel containing 7 M urea, and stained with 10 μg/ml EtBr.

Construction of Recombinant Adenoviruses Coding MMP-13 Antisense and Sense Ribozymes

Replication deficient (E1- and E3-) adenoviruses harboring MMP-13 antisense and sense ribozymes were constructed, as previously described (Ala-aho et al. 2002b). The corresponding double stranded DNA molecules were subcloned into pCA3 shuttle vector digested with EcoRI and XbaI under the control of CMV IE promoter. Adenoviral genomic plasmid pBHG10 and the shuttle vectors containing ribozyme coding region were co-transfected into 293 cells (all from Microbix Biosystems, Toronto, ON) with CalPhosMaximizer kit (Clontech, Palo Alto, Calif.). After three weeks, plaques were visible and cell layer was harvested in PBS containing 10% glycerol and viruses were released from cells with freon extraction and subjected to plaque purification in 96 well plates. Positive recombinants were identified by PCR and sequencing using recombinant clone viral DNA as template with pCA3 vector specific oligonucleotide primers pCA3seq3 (5′-CATCCACGCTGTTTTGACC-3′) and pCA3seq5 (5′-GAAATTTGTGATGCTATTGC-3′). Positive clones of recombinant adenovirus (RAdMMP-13ASRz and RAdMMP-13senseRz) were chosen to generate high titer preparation by freon extraction, cesium chloride banding and dialysis (Ala-aho et al. 2002b). Determination of viral titer was conducted as described previously (Lu et al. 1998).

Adenoviral Cell Infections

The multiplicity of infection (MOI) for obtaining maximal infection efficiency of UT-SCC-7 cells has been determined previously (Ala-aho et al. 2002a). The MOI for obtaining maximal infection efficiency of HaCaT and A-5 cell lines was determined using recombinant adenovirus RAdLacZ, which contains the Escherichia coli beta-galactosidase gene (lacZ) under the control of CVM IE promoter (Wilkinson and Akrigg 1992) (kindly provided by Dr. Gavin W. G. Wilkinson, University of Cardiff, Wales). Cells (6×10⁵) were plated, RAdLacZ was added to cultures at different MOI on the following day, cultures were incubated for 6 h, washed with PBS, and maintained for 16 h in DMEM containing 0.5% FCS. The cells were then fixed and stained for beta-galactosidase activity, as described previously (Ravanti et al. 1999b). In experiments, cells were infected with recombinant adenoviruses at MOI 700 for UT-SCC-7 cells, or at MOI 500 for HaCaT and A-5 cells, incubated for 6 h in DMEM with 0.5% FCS. The medium was changed and incubations were continued for 24 h prior to invasion assays or 24-96 h prior to determination of cell viability.

Invasion Assays

Cell culture inserts (Falcon 3097, Becton Dickinson) with 8.0 μm pore size were coated with 25 μg of reconstituted basement membrane (Matrigel, Becton Dickinson), as described previously (Ala-aho et al. 2000). For invasions, cells (2×10⁵/chamber) suspended in DMEM containing 0.1% BSA were placed on top of the gel in the upper chamber in a final volume of 200 μl, with DMEM (700 μl) containing 10% FCS as chemoattractant in the lower chamber. After 24 h, cells on the upper surface were gently removed with a cotton bud and the invaded cells on the lower surface were fixed in 2% paraformaldehyde, counterstained with 0.1% crystal violet, and counted.

RT-PCR

Total RNA was isolated from tumors using the Qiagen RNeasy kit (Qiagen, Chatsworth, Calif.). The expression of MMP-13 mRNA in SCC xenografts was determined by RT-PCR. Aliquots of total RNA (100 ng) were reverse transcribed into cDNA and a 300 bp fragment of human MMP-13 cDNA corresponding to nucleotides 534-833 was amplified by PCR using forward oligonucleotide (5′-CATTTGATGGGCCCTCTGGCCTGC-3′) and reverse oligonucleotide (5′-GTTTAGGGTTGGGGTCTTCATCTC-3′) as described previously (Ravanti et al. 1999a). The forward oligonucleotide (5′-CCCATGGCAAATTCCATGGCA-3′) and reverse oligonucleotide (5′-TCTAGACGGCAGGTCAGGTC-3′) was used to amplify glyceraldehyde-3-phosphate dehydrogenase (GAPDH) as a housekeeping gene control with 40 cycles of denaturation at 94° C., annealing at 66° C., and extension at 72° C. The generated products were subjected to electrophoresis on a 2% agarose gel and were visualized by ethidium bromide staining.

Assay of MMP-13 and MMP-1 Production

The production of MMP-13 and MMP-1 by SCC cells was determined by Western blot analysis, as described previously (Ala-aho et al. 2000) using monoclonal antibody (181-15A12) against human MMP-13 (Calbiochem, San Diego, Calif.) in dilution 1:100 and rabbit polyconal antibody against human MMP-1 (kindly provided by Dr. H. Birkedal-Hansen, NIDR, Bethesda, Md.) in dilution 1:5000, followed by detection of specifically bound primary antibodies with peroxidase-conjugated secondary antibodies and visualized by enhanced chemiluminescence (ECL; Amersham). For TIMP-1 analysis, aliquots of conditioned media were reduced with 5% beta-mercaptoethanol prior to electrophoretic fractionation and analyzed with polyclonal rabbit antibody (Chemicon International Inc., Temecula, Calif.) in dilution of 1:1000.

Gelatin Zymography

Aliquots of conditioned media were fractionated on 10% SDS-PAGE containing 1 mg/ml gelatin (G-9382; Sigma) and 0.5 mg/ml 2-methoxy-2,4-diphenyl-3(2H)-furanone (Fluka 645989) (O'Grady et al. 1984). The gels were washed for 30 min in 50 mM Tris, 0.02% NaN₃ and 2.5% Triton X-100, pH 7.5 and for 30 min in the same buffer supplemented with 5 mM CaCl₂ and 1 mM ZnCl₂ (Heussen and Dowdle 1980). The gels were then incubated in 50 mM Tris, 0.02% NaN₃, 5 mM CaCl2 and 1 mM ZnCl₂ for 24 h at 37° C., fixed in 50% methanol/7% acetic acid, stained with 0.2% Coomassie Blue G250 and photographed as previously described (Ala-aho et al. 2000).

Determination of Viable Cell Number

For cell viability assays 1,5×10⁴ cells were seeded on 96 well plates and infected with recombinant adenovirus RAdMMP-13ASRz, RAdMMP-13senseRz, or with corresponding empty control adenovirus RAdpCA3 at MOI 700 for 6 hours. The cells were incubated for different periods of time and the number of viable cells was determined by CellTiter 96™ AQueous Non-Radioactive Cell Proliferation Assay (Promega, Madison, Wis.) according to manufacturer's instructions. The number of viable cells were compared to uninfected cells on corresponding incubation time.

UT-SCC-7 cells were seeded on 35 mm plates (2×10⁴ cell/plate) and infected with recombinant adenoviruses as described above and cultured in 0.5% FCS in DMEM for different periods of time. Cells were trypsinized and counted from three plates in each time point.

Immunostaining of SCC Cells

Adenovirus infected SCC cells were cultured in the serum-free DMEM on glass slides for different periods of times, washed with PBS, fixed with ice-cold methanol, and washed with PBS. To detect apoptotic cells, the TUNEL reaction was performed using the In Situ Cell Death Detection Kit (Roche, Germany) according to the manufacturer's instruction. In parallel cultures, the nuclei of SCC cells were stained with Hoechst-33342 (10 μg/ml), analyzed by fluorescence microscopy and photographed for detection of apoptotic cells.

Growth of SCC Zenografts in SCID/SCID Mice

All experiments with mice were performed according to institutional animal care guidelines and with permission of the animal test review board of the University of Turku, Finland. Six to eight weeks old severe combined immunodeficiency (SCID/SCID) mice were used in all experiments. In ex vivo experiments UT-SCC-7 cells were infected as described above at MOI 700, incubated for 6 h, washed with PBS, and detached with trypsin. Trypsin was neutralized with 10% FCS in DMEM and cells (5×10⁶/mouse) in 100 μl of PBS were injected subcutaneously to the back of SCID mice. Each experimental group contained 5 male mice. Tumor size was measured twice a week and calculated as length ×width²×0.5.

For intratumoral injection of recombinant adenoviruses, tumors were established by injecting 5×10⁶ UT-SCC-7 cells subcutaneously to back of mice and allowing tumors grow 100 mm³. 1×10⁹ pfu of the recombinant adenovirus in 0.1 ml PBS or PBS only was injected intratumorally 2-3 times a week for three weeks. Tumor size was measured before each injection and calculated as above.

Immunohistochemistry

Tumors were fixed overnight in phosphate buffered 10% formalin and embedded in paraffin for histologic assessment. Serial sections of 5 μm were taken from each paraffin-embedded tissue block for immunohistochemistry. Deparaffinized sections were processed with citrate buffer in microwave oven. MMP-13 immunostaining was performed as described earlier using monoclonal antibody against human MMP-13 (181-15A12; Calbiochem, San Diego, Calif.), which does not cross-react with mouse MMP-13 (Ravanti et al. 2001). Negative control sections were incubated without primary antibody.

Mayer's hematoxylin was used as counterstain in all immunostainings. Ki67 were determined immunohistochemically on paraffin embedded sections using monoclonal antibody against human Ki67 (MIB-1, DAKO, Denmark). Relative number of Ki67 positive cells were determined using Soft Imaging System's analySIS® program.

In situ Gelatin Zymography

For in situ zymography pieces of tumors were mounted into Tissue-Tek and flash-frozen in liquid isopentane. Gelatinase activity was deteced by gelatin in situ zymography as described previously (George et al. 2000). Briefly, 7 μm frozen sections (4 sections/sample) were applied to glass slides and coated with LM-1 photographic emilsion (Amersham International, UK) diluted 1:2 with incubation medium (50 mM Tris-HCl, 50 mM NaCl, 10 mM CaCl₂, 0.05% Brij 35, pH 7.6). After incubation overnight at 37° C. in a humified box, slides were developed in the light with Kodak D-19 developer (Kodak, Bridgend, Wales, UK) and fixed using Kodak Unifix solution. In addition gelatinase zymography for a tumor sample treated with RAdMMP-13senseRz was performed with 500 nM of the MMP inhibitor BB-94 (Pfizer, Sandwich, UK). Gelatinolytic activity was identified as white areas of lysis on the black background.

### Results

In vitro Cleavage of Human MMP-13 mRNA by MMP-13 Antisense Ribozyme

A antisense MMP-13 hammerhead ribozyme was designed to cleave human MMP-13 mRNA between nucleotides 716 and 717 (FIG. 1 A). The homology search of human genome sequences revealed no homology to other known human or mouse genes. The homology regions, binding arms, flanking the catalytic ribozyme structure to the target mRNA are 9 and 8 nucleotides on the 5′ and 3′ ends, respectively. As a control we also designed a sense ribozyme containing the hammerhead catalytic loop but unable to anneal to MMP-13 or any other known mRNA (FIG. 1 A). The MMP-13 antisense and sense sequences containing hammerhead ribozyme sequence were cloned into pCl-neo vector and transcribed using T7 RNA polymerase. MMP-13 antisense ribozyme was then tested for its ability to cleave human MMP-13 mRNA in vitro. The cleavage of MMP-13 mRNA by antisense ribozyme resulted in generation of fragments of 706 and 736 nucleotides length, as expected (FIG. 1 B,C). After 60 min incubation 50% of target RNA was cleaved and after 8 h all MMP-13 RNA was cleaved into two fragments. No cleavage of MMP-13 mRNA was seen with sense ribozyme.

### Adenoviral Delivery of MMP-13 Antisense Ribozyme Inhibits MMP-13 Expression and Invasion of Squamous Carcinoma Cells

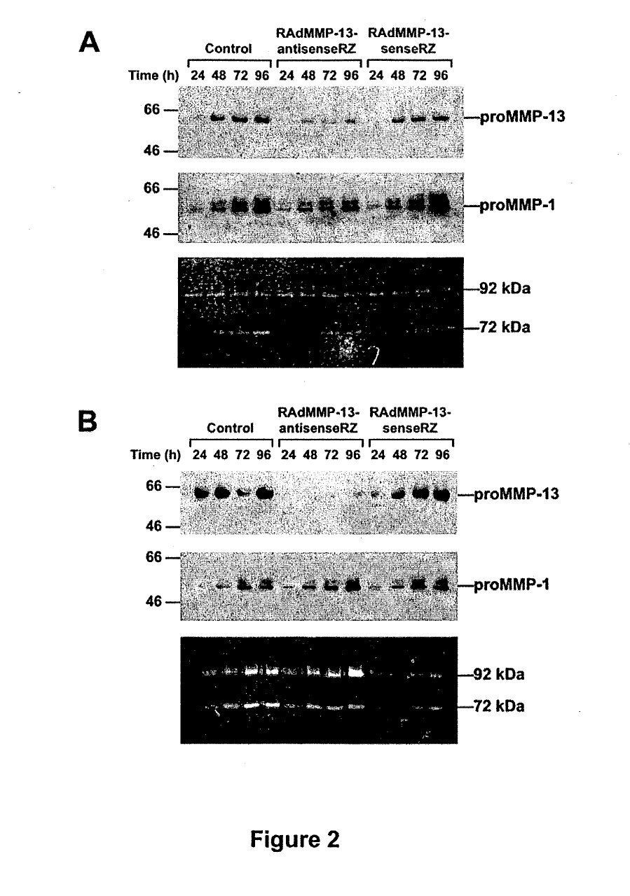

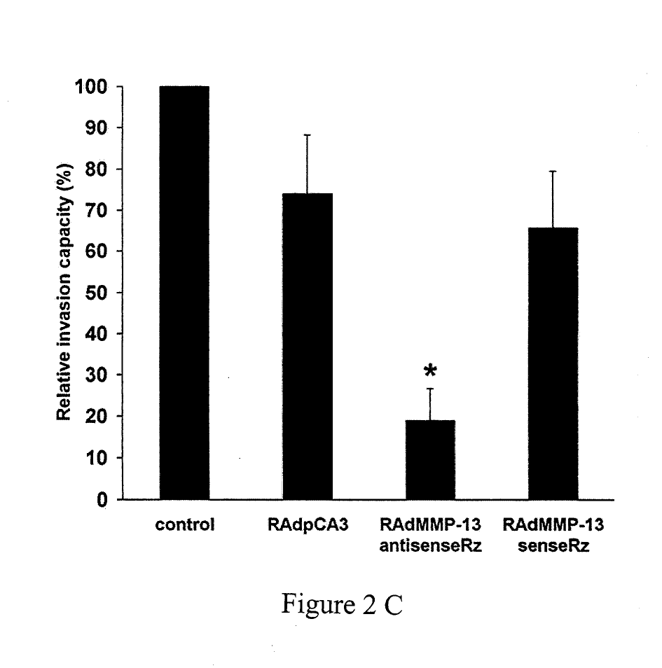

Squamous cell carcinomas (SCCs) of the head and neck are tumors with high invasion capacity and they express high levels of MMP-13 (Johansson et al. 1997a). To examine the role of MMP-13 in SCC cell invasion, we constructed a recombinant adenovirus RAdMMP-13asRz encoding MMP-13 antisense ribozyme and used it to transduce SCC cells. Adenovirus-mediated expression of MMP-13 antisense ribozyme resulted in potent inhibition in MMP-13 production noted 24 h after adenoviral infection of UT-SCC-7 cells (FIG. 2 A). In the same cells, production of MMP-1 was not markedly suppressed after infection with RAdMMP-13ASRz. Furthermore, MMP-13 antisense ribozyme had no effect on the production of 92-kDa-gelatinase (MMP-9) and 72-kDa gelatinase (MMP-2) by these cells (FIG. 2 A). Infection of cells with control adenovirus encoding MMP-13 sense ribozyme had no effect on MMP-13 production. The effect of adenoviral delivery of MMP-13 antisense ribozyme on the expression of MMP-13 was also examined in HaCaT keratinocytes and ras-transformed HaCaT cells (Boukamp et al. 1990), both expressing MMP-13. In both cell lines marked inhibition of MMP-13 production in response to MMP-13 antisense ribozyme was noted (FIG. 2 B and data not shown), whereas production of MMP-1 and 92-kDa and 72-kDa gelatinases by these cells were not altered.

As compared to other collagenolytic MMPs, MMP-13 potently degrades components of basement membranes (Knäuper et al. 1996). Accordingly, we have noted, that the expression of MMP-13 enhances invasion of malignant cells through reconstituted basement membrane, Matrigel (Ala-aho et al. 2002b). As RAdMMP-13ASRz potently inhibits the expression of MMP-13, we examined its effect on the invasion of SCC cells through Matrigel. As shown in FIG. 2 C, invasion of UT-SCC-7 cells was significantly (by 80%) inhibited by MMP-13 antisense ribozyme, whereas infection of UT-SCC-7 cells with control virus RAdMMP-13senseRz had no effect on the invasion capacity of UT-SCC-7 cells. These results shows that MMP-13 antisense riboxyme inhibits the invasion capacity of SCC cells, most likely due to suppression in the expression of MMP-13.

MMP-13 Antisense Ribozyme Suppresses Squamous Carcinoma Cell Growth and Induces Apoptosis

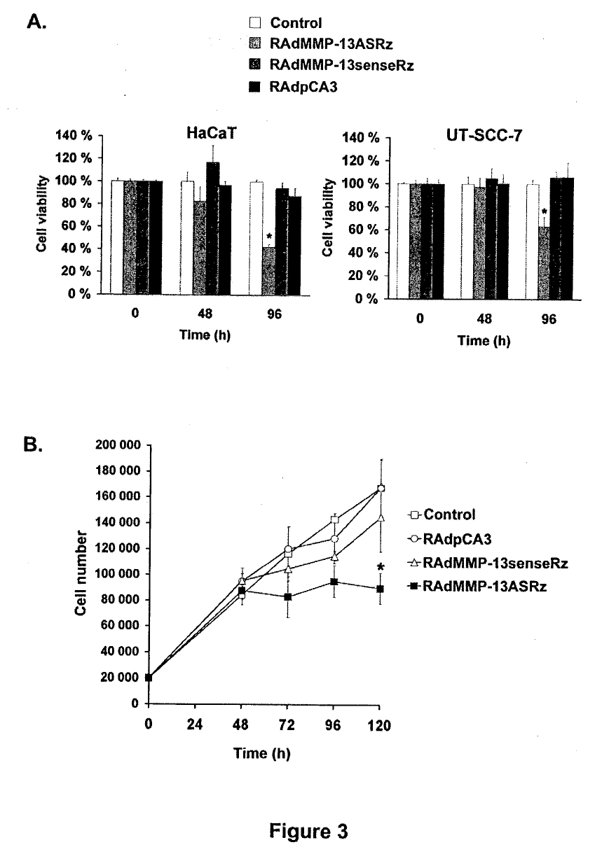

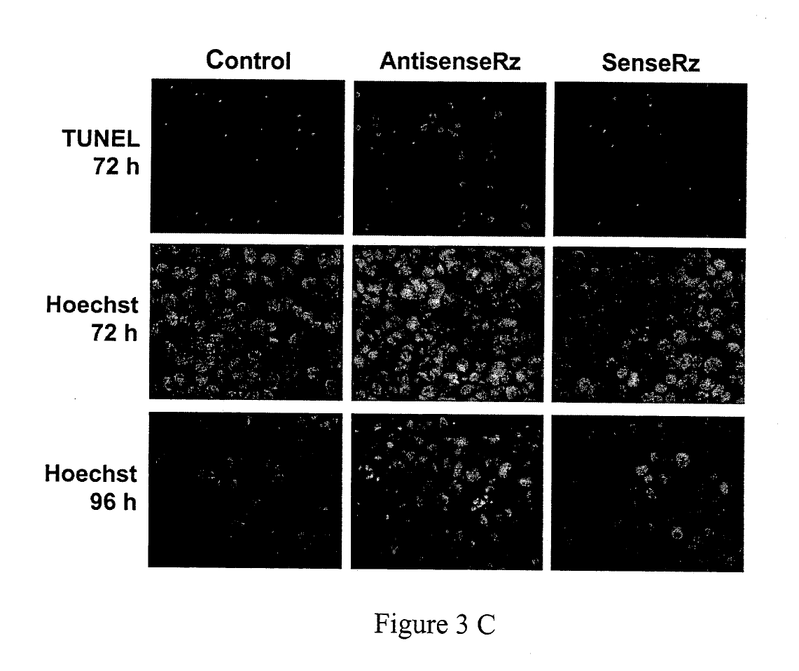

To test the effects of RAdMMP-13ASRz on squamous carcinoma cell growth in vitro, we transduced UT-SCC-7 cells with RAdMMP-13ASRz at MOI 700 and determined the number of viable cells. The MMP-13 antisense virus reduced the number of viable UT-SCC-7 cells significantly at 96 h after the infection while sense adenovirus had no effect on cell growth in comparison with uninfected control cells (FIG. 3 A, right panel). Similar results were obtained with HaCaT keratinocytes (FIG. 3 A, left panel). To determine the effect of MMP-13 antisense ribozyme on cell growth rate, 2×10⁴ UT-SCC-7 cells were cultured in 35 mm dishes and transduced by adenoviruses RAdpCA3, RAdMMP-13ASRz and RAdMMP-13senseRz. Number of cells were counted from individual dishes every 24 h beginning at day 2. RAdMMP-13ASRz inhibited growth of UT-SCC-7 cells if compared to uninfected cells (FIG. 3 B). Infecting of cells with control sense virus had no marked effect on cell proliferation.

To further elucidate mechanism of reduction in UT-SCC-7 cell growth we labeled the DNA of SCC cells with TUNEL reaction at different periods of times. The incorporated label into nucleotides was detected in SCC cells three days after adenoviral delivery of MMP-13 antisense ribozyme while uninfected or RAdMMP-13senseRz infected cultures showed few TUNEL positive cells (FIG. 3 C). The adenovirus infected cells were also stained with Hoechst to reveal cells with condensed nuclei as a marker of apoptosis. Furthermore, release of apoptotic bodies was seen in RAdMMP-13ASRz infected SCC cells at day four after infection (FIG. 3 C).

MMP-13 Antisense Ribozyme Inhibits Implantation and Growth of Squamous Cell Carcinoma in SCID Mice

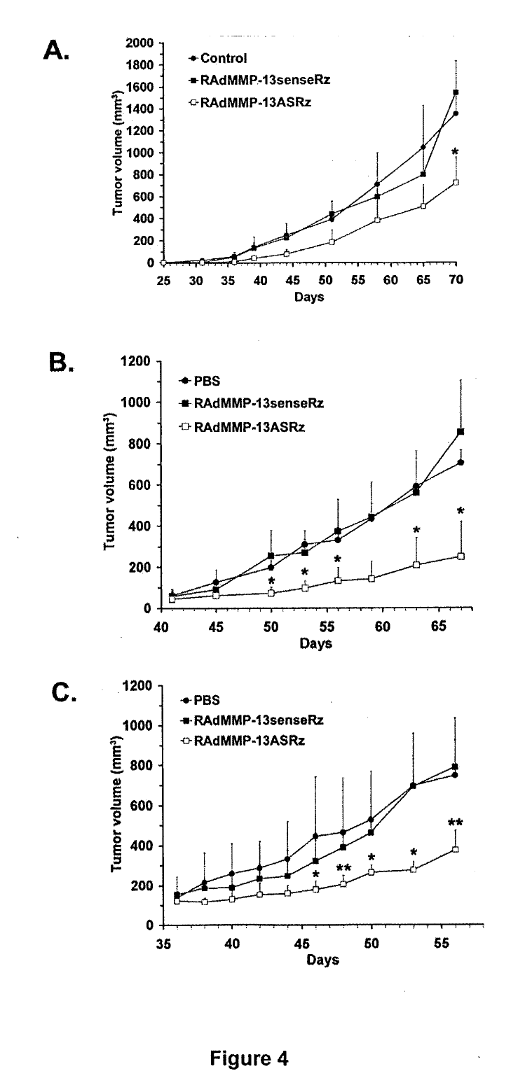

To examine whether MMP-13 also plays a role in SCC cell survival and invasion in vivo, we infected UT-SCC-7 cells with 700 MOI of RAdMMP-13ASRz or RAdMMP-13senseRz. Following day of transduction, 5×10⁶ UT-SCC-7 cells were inoculated subcutaneously into the back of SCID mice. Tumor size was measured by twice a week. Squamous cell carcinoma formation was significantly slover by RAdMMP-13ASRz in comparison with control cells and RAdMMP-13senseRz infected cells (FIG. 4 A).

Next, we wanted to examine the therapeutic efficacy of RAdMMP-13ASRz on established tumors. 5×10⁶ UT-SCC-7 cells were inoculated subcutaneously into the back of SCID mice and tumor size was measured three times a week. In the first experiment, implanted SCC tumors reach a size of 100 mm³ six weeks after tumor cell intake. The recombinant adenovirus was administered intratumorally twice a week for 4 weeks starting on day 41. Treatment of SCC tumors with MMP-13ASRz resulted in inhibition of tumor growth (FIG. 4 B). At day 67 tumor size was 38% of control PBS treated tumors. RAdMMP-13senseRz had no effect on tumor growth. The experiment was repeated but virus administration was initiated at day 36 when the tumors reached approximately the size of 100 mm³ and the tumors were then inoculated with recombinant adenoviruses three times a week for three weeks. Again, SCC tumor growth was inhibited significantly (by 50%) by RAdMMP-13ASRz while RAdMMP-13senseRz had no effect on tumor growth when compared to PBS treated tumors (FIG. 4 C).

MMP-13 Antisense Ribozyme Inhibits Expression of MMP-13 and Gelatinolytic Activity in Squamous Cell Carcinomas in vivo

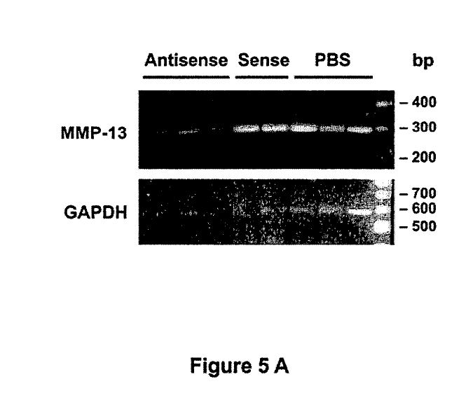

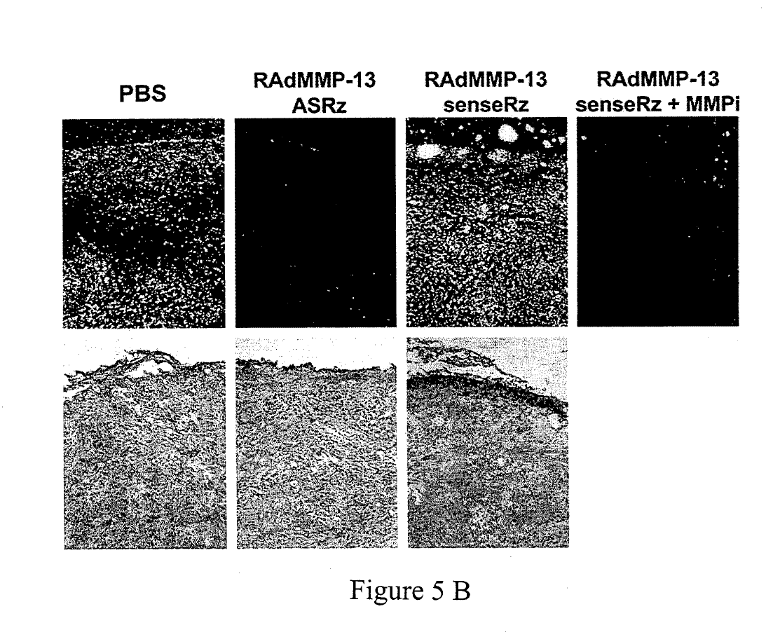

To test the inhibitory effect of MMP-13 antisense ribozyme on MMP-13 expression, RT-PCR was performed from the RNA samples isolated from tumor tissue. Tumors infected with RAdMMP-13ASRz showed decrease in MMP-13 mRNA levels as compared to RAdMMP-13senseRz infected or PBS injected tumors indicating that MMP-13 antisense ribozyme decreased MMP-13 expression in vivo (FIG. 5 A).

To verify the inhibitory effect of adenovirally delivered MMP-13 antisense ribozyme on MMP activity in vivo, tumor sections were studied by in situ gelatin zymography. Marked gelatinase activity was observed at PBS and RAdMMP-13senseRz treated tumors (FIG. 5 B). Potent reduction in gelatinolytic activity was observed in RAdMMP-13ASRz injected tumors. Addition of MMP inhibitor BB-94 totally blocked the gelatinase activity in tumor tissue confirming that MMPs are involved in gelatin degradation and that the inhibition on RAdMMP-13ASRz treated tumors is due to inhibition of MMP activity.

MMP-13 Antisense Ribozyme Reduces Number of Proliferating Cells in Squamous Cell Carcinoma in SCID Mice

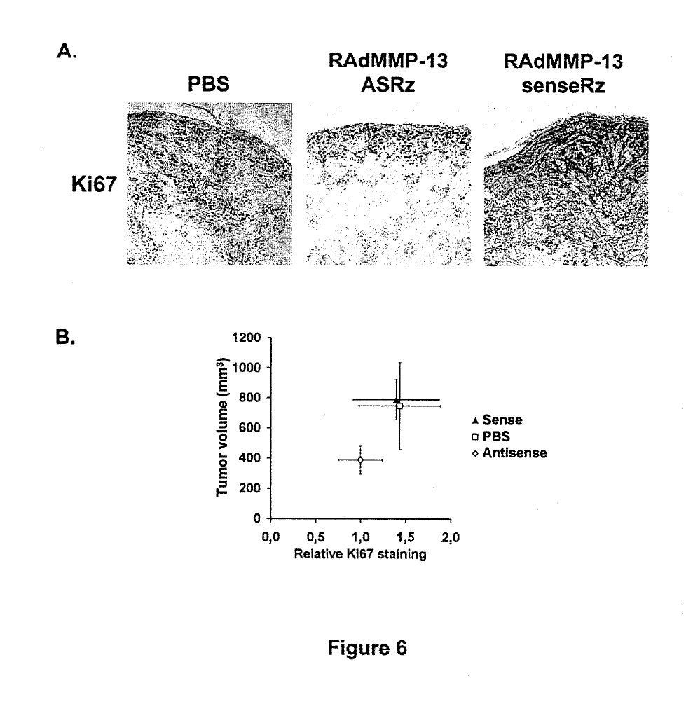

Proliferating cells were determined using Ki67 as a marker of proliferation rate in tumor sections. The Ki67 positive cells were determined near the front of tumor tissue (FIG. 6 A). The Ki67 positive area was equal in the PBS injected and RAdMMP-13senseRz injected tumors. Interestingly, the amount Ki67 positive cells in tumor treated with MMP-13 antisense ribozyme was 70% of the control groups (FIG. 6 B).

Additional Examples of Effective MMP-13 Antisense Ribozymes

Three alternative MMP-13 antisense ribozymes was designed to recognize and cleave MMP-13 mRNA. The ribozymes are named according to their cleavage site. Target sequence indicates the corresponding nucleotides at the human MMP-13 mRNA sequence.

| Ribozyme | Target sequence | Cleavage site |
|---|---|---|
| Rz80 | 72-88 | 80-81 |
| Rz369 | 361-377 | 369-370 |
| Rz430 | 422-438 | 430-431 |

For comparison, the antisense ribozyme shown in FIG. 1 A (Rz716) can be disclosed as follows:

| | | |
|---|---|---|
| Rz716 | 707-724 | 716-717 |

Sequences of the three additional MMP-13 antisense ribozymes.

The following oligonucleotides were used for cloning MMP-13 ribozyme expression vectors. The flanking restriction enzyme cleavage sites are underlined.

| | |
|---|---|
| Rz80frw | 5′-GAATTCAGGGCCCCTGATGAGTCCGTGAGGACGAAACAATGAGTCTAGA-3′ |
| Rz80rev | 5′-TCTAGACTCATTGTTTCGTCCTCACGGACTCATCAGGGGCCCTGAATTC-3′ |
| Rz369frw | 5′-GAATTCATTTTGCTGATGAGTCCGTGAGGACGAAACCATTTATCTAGA-3′ |
| Rz369rev | 5′-TCTAGATAAATGGTTTCGTCCTCACGGACTCATCAGCAAAATGAATTC-3′ |
| Rz430frw | 5′-GAATTCGCCTTTTCCTGATGAGTCCGTGAGGACGAAACTTCAGATCTAGA-3′ |
| Rz430rev | 5′-TCTAGATCTGAAGTTTCGTCCTCACGGACTCATCAGGAAAAGGCGAATTC-3′ |

Effectiveness of the additional ribozymes as compared to Rz716.

In vitro cleavage efficiency was determined as previously described. Human MMP-13 mRNA generated by in vitro transcription was incubated with antisense ribozymes for 5 h and analyzed by electrophoresis on agarose gel and visualized by ethidium bromide. In vitro cleavage efficiency was compared to that of Rz716, i.e. the antisense ribozyme of FIG. 1 A, which cleaved MMP-13 transcript most efficiently.

| Ribozyme | Efficiency compared to Rz716 |
|---|---|
| Rz80 | 4% |
| Rz369 | 93% |
| Rz40O | 37% |
| Rz716 | 100% |

Discussion

In the present study, we have designed an MMP-13 antisense ribozyme and tested the efficacy of adenovirus mediated transfer of ribozyme on the growth of squamous cell carcinomas in SCID mice. Based to homology search, the MMP-13 antisense ribozyme does not recognize mouse or other human genes. The MMP-13 antisense ribozyme specifically cleaves the MMP-13 transcript in a cell-free system and adenovirus mediated transfer of ribozyme results in potent inhibition of MMP-13 expression by different cell lines in culture. We have also shown that this reduced expression of MMP-13 suppresses growth of squamous cell carcinoma xenografts in SCID mice.

Specific inhibition of particular MMP overexpression in cancer or pathological conditions by antisense ribozyme may serve useful tools for efficient gene therapy. A ribozyme targeted to MMP-9 have been shown to inhibit metastasis of rat sarcomas (Hua and Muschel 1996) and ribozyme against MMP-3 inhibits MMP-3 mRNA expression in articular cartilage explants (Jarvis et al. 2000). Different MMPs are overexpressed in various tumors and therefore the appropriate targets for therapeutic intervention may vary in each type of tumor.

Human collagenase-3 (MMP-13) is not expressed by normal epidermal keratinocytes in culture or in vivo (Johansson et al. 1997c; Vaalamo et al. 1997), but it is expressed by malignantly transformed epidermal keratinocytes, i.e. squamous carcinoma cells in culture and in vivo (Johansson et al. 1997a; Johansson et al. 1999). However, no expression of MMP-13 is noted in premalignant tumors in human skin. These observations show, that MMP-13 expression serves as a marker for transformation of squamous epithelial cells and suggest a marked role for MMP-13 in invasion of SCC cells. Furthermore, previous observations by us and others have shown, that MMP-13 is specifically expressed by tumor cells at the invading edge of SCCs of the head and neck and vulva (Airola et al. 1997; Cazorla et al. 1998; Johansson et al. 1997a; Johansson et al. 1999). The inhibition of MMP-13 expression in invasive transformed human epidermal keratinocytes by IFN-gamma or p53 markedly reduces their invasion capacity (Ala-aho et al. 2002a; Ala-aho et al. 2000). In addition, we have shown that expression of MMP-13 by invasive HT-1080 cell line increases their invasion capacity through type I collagen and Matrigel (Ala-aho et al. 2002b). Together these features make MMP-13 a tempting target for therapy aimed at inhibiting growth and invasion of SCCs.

Marked inhibition of MMP-13 production in SCC cells was seen within 24 h after adenoviral delivery of MMP-13 ribozyme, whereas no reduction in cell viability was detected during first 48 hours. Furthermore, no apoptotic cells were detected within the first 24 h after adenoviral delivery of MMP-13 antisense ribozyme. Together these observations indicate that MMP-13 antisense ribozyme inhibits MMP-13 gene expression independently of its ability to induce apoptosis. In addition, MMP-13 antisense ribozyme inhibit SCC cell invasion through Matrigel within the first 24 h after adenoviral transduction indicating that invasion is inhibited due to the reduction of MMP-13 expression rather that reduction in cell viability. The condensation of nuclei and induction of apoptosis was detected in SCC cells 72 h after adenoviral delivery of MMP-13 antisense ribozyme. Apoptotic condensation of the SCC cell nuclei was detected 24 h later and marked inhibition on cell growth or viability was detected 96 and 120 hours after adenoviral delivery of MMP-13 antisense ribozyme. Together these data suggest that suppression of MMP-13 expression by antisense ribozyme results in inhibition of SCC cell growth and survival by apoptosis.

The adenovirus mediated gene delivery results in relatively short term expression of the transgene, since it is not permanently targeted into host cell genome, and is lost during cell division. We found that tumor implantation was delayed in SCID mice by a single infection of MMP-13 antisense ribozyme into SCC cells. Interestingly, one of the five mice injected with RAdMMP-13ASRz infected cells, generated no tumor.

The infection of tumor xenografts with RAdMMP-13ASRz resulted in suppression of tumor growth. However, the increased dose of adenoviral infection from twice a week to three times a week did not increase the inhibitory effect of MMP-13 antisense ribozyme. This may be due the limited transduction efficiency. The estimated efficiency of adenoviral transduction into SCC tumors by single injection is about 3%.

The results reported here demonstrate for the first time the therapeutic efficiancy specific inhibition of MMP-13 expression by MMP-13 antisense ribozyme in SCC growth in vivo. In our models, the tumor growth was clearly suppressed by adenoviral delivery of MMP-13 antisense ribozyme into SCID mice. The adenoviral-based approach may have only minor clinical utility in the local tumors and the cases in which the limited treatment options currently exist. For succesful applications more improved delivery approaches to mediate high-level expression of ribozyme, will be required. Currently the viral vectors best suited for ribozyme delivery are adenoassociated viruses (AAV) which leads to long-term genenic transduction of infected cells (Hernandez et al. 1999). However, the virus based applications have a limited infection efficiency. Another approach is the direct delivery of ribozyme molecules to tissues and this has led to development of nuclease-resistant ribozymes because of the short half-life of RNA. The nuclease-resistant chemically synthetized ribozymes can be administered subcutaneously or intravenously and they have excellent specificity and they are well tolerated (Usman and Blatt 2000). The nuclease-resistant ribozymes targeted against VEGF receptor mRNA has shown to decrease lung metastases in a dose-dependent manner (Pavco et al. 2000). Inhibition of MMP activity in the extracellular space has been studied as an approach to inhibit growth and invasion of neoplastic cells. Several broad-range MMP inhibitors have shown efficiency against malignant tumors in preclinical studies (Nelson et al. 2000). They have been tested in clinical trials in patients with different types of tumors, but the outcome from these studies have been disappointing.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### REFERENCES

Airola, K., Johansson, N., Kariniemi, A.-L., Kähäri, V.-M., Saarialho-Kere, U. (1997) Human collagenase-3 is expressed in malignant squamous epithelium of the skin. J. Invest. Dermatol. 109:225-231.

Airola, K., Karonen, T., Vaalamo, M., Lehti, K., Lohi, J., Kariniemi, A. L., Keski-Oja, J., Saarialho-Kere, U. K. (1999) Expression of collagenases-1 and -3 and their inhibitors TIMP-1 and -3 correlates with the level of invasion in malignant melanomas. Br. J. Cancer 80:733-743.

Ala-aho, R., Grénman, R., Seth, P., Kähäri, V.-M. (2002a) Adenoviral delivery of p53 gene suppresses expression of collagenase-3 (MMP-13) in squamous carcinoma cells. Oncogene 21:1187-1195.

Ala-aho, R., Johansson, N., Baker, A. H., Kähäri, V.-M. (2002b) Expression of collagenase-3 (MMP-13) enhances invasion of human fibrosarcoma HT-1080 cells. Int. J.Cancer 97:283-289.

Ala-aho, R., Johansson, N., Grénman, R., Fusenig, N. E., López-Otín, C., Kähäri, V.-M. (2000) Inhibition of collagenase-3 (MMP-13) expression in transformed human keratinocytes by interferon-γ is associated with activation of extracellular signal-regulated kinase-1,2 and STAT1. Oncogene 19:248-257.

Ashworth, J. L., Murphy, G., Rock, M. J., Sherratt, M. J., Shapiro, S. D., Shuttleworth, C. A., Kielty, C. M. (1999) Fibrillin degradation by matrix metalloproteinases: implications for connective tissue remodelling. Biochem. J. 340:171-181.

Boström, P. J., Ravanti, L., Reunanen, N., Aaltonen, V., Söderström, K.-O., Kähäri, V.-M., Laato, M. (2000) Expression of collagenase-3 (matrix metalloproteinase-13) in transitional cell carcinoma of the urinary bladder. Int. J. Cancer 88:417-423.

Boukamp, P., Stanbridge, E. J., Foo, D. Y., Cerutti, P. A., Fusenig, N. E. (1990) c-Ha-ras oncogene expression in immortalized human keratinocytes (HaCaT) alters growth potential in vivo but lacks correlation with malignancy. Cancer Res. 50:2840-2847.

Cazorla, M., Hernandez, L., Nadal, A., Balbín, M., López, J. M., Vizoso, F., Fernandez, P. L., Iwata, K., Cardesa, A., López-Otín, C., Campo, E. (1998) Collagenase-3 expression is associated with advanced local invasion in human squamous cell carcinomas of the larynx. J. Pathol. 186:144-150.

Etoh, T., Inoue, H., Yoshikawa, Y., Barnard, G. F., Kitano, S., Mori, M. (2000) Increased expression of collagenase-3 (MMP-13) and MT1-MMP in oesophageal cancer is related to cancer aggressiveness. Gut 47:50-56.

Fosang, A. J., Last, K., Knäuper, V., Murphy, G., Neame, P. J. (1996) Degradation of cartilage aggrecan by collagenase-3 (MMP-13). FEBS-Lett. 380:17-20.

George, S. J., Lloyd, C. T., Angelini, G. D., Newby, A. C., Baker, A. H. (2000) Inhibition of late vein graft neointima formation in human and porcine models by adenovirus-mediated overexpression of tissue inhibitor of metalloproteinase-3. Circulation 101:296-304.

Heppner, K. J., Matrisian, L. M., Jensen, R. A., Rodgers, W. H. (1996) Expression of most matrix metalloproteinase family members in breast cancer represents a tumor-induced host response. Am. J. Pathol. 149:273-282.

Hernandez, Y. J., Wang, J., Kearns, W. G., Loiler, S., Poirier, A., Flotte, T. R. (1999) Latent adeno-associated virus infection elicits humoral but not cell-mediated immune responses in a nonhuman primate model. J. Virol. 73:8549-8558.

Heussen, C., Dowdle, E. B. (1980) Electrophoretic analysis of plasminogen activators in polyacrylamide gels containing sodium dodecyl sulfate and copolymerized substrates. Anal. Biochem. 102:196-202.

Hua, J., Muschel, R. J. (1996) Inhibition of matrix metalloproteinase 9 expression by a ribozyme blocks metastasis in a rat sarcoma model system. Cancer Res. 56:5279-5284.

Jarvis, T. C., Bouhana, K. S., Lesch, M. E., Brown, S. A., Parry, T. J., Schrier, D. J., Hunt, S. W., 3rd, Pavco, P. A., Flory, C. M. (2000) Ribozymes as tools for therapeutic target validation in arthritis. J. Immunol. 165:493-498.

Johansson, N., Ahonen, M., Kähäri, V.-M. (2000) Matrix metalloproteinases in tumor invasion. Cell. Mol. Life Sci. 57:5-15.

Johansson, N., Airola, K., Grénman, R., Kariniemi, A.-L., Saarialho-Kere, U., Kähäri, V.-M. (1997a) Expression of collagenase-3 (matrix metalloproteinase-13) in squamos cell carcinomas of the head and neck. Am. J. Pathol. 151:499-508.

Johansson, N., Saarialho-Kere, U., Airola, K., Herva, R., Nissinen, L., Westermarck, J., Vuorio, E., Heino, J., Kähäri, V.-M. (1997b) Collagenase-3 (MMP-13) is expressed by hypertrophic chondrocytes, periosteal cells, and osteoblasts during human fetal bone development. Dev. Dyn. 208:387-395.

Johansson, N., Vaalamo, M., Grénman, S., Hietanen, S., Klemi, P., Saarialho-Kere, U., Kähäri, V.-M. (1999) Collagenase-3 (MMP-13) is expressed by tumor cells in invasive vulvar squamous cell carcinomas. Am. J. Pathol 154:469-480.

Johansson, N., Westermarck, J., Leppäa, S., Häkkinen, L., Koivisto, L., López-Otín, C., Peltonen, J., Heino, J., Kähäri, V.-M. (1997c) Collagenase 3 (matrix metalloproteinase 13) gene expression by HaCaT Keratinocytes is enchanced by tumor necrosis factor-α and transforming growth factor-β. Cell. Growth. Differ. 8:243-250.

Knäuper, V., Cowell, S., Smith, B., López-Otín, C., O'Shea, M., Morris, H., Zardi, L., Murphy, G. (1997) The role of the C-terminal domain of human collagenase-3 (MMP-13) in the activation of procollagenase-3, substrate specificity, and tissue inhibitor of metalloproteinase interaction. J.Biol. Chem. 272:7608-7016.

Knäuper, V., López-Otín, C., Smith, B., Knight, G., Murphy, G. (1996) Biochemical characterization of human collagenase-3. J. Biol. Chem. 271:1544-1550.

Kähäri, V.-M., Saarialho-Kere, U. (1997) Matrix metalloproteinases in skin. Exp. Dermatol.: 199-213.

Lindy, O., Konttinen, Y. T., Sorsa, T., Ding, Y., Santavirta, S., Ceponis, A., López-Otín, C. (1997) Matrix metalloproteinase 13 (collagenase 3) in human rheumatoid synovium. Arthritis. Rheum. 40:1391-1399.

Lu, Y., Zhang, Y., Steiner, M. S. (1998) Efficient identification of recombinant adenoviruses by direct plaque screening. DNA Cell Biol. 17:643-645.

Mao, D., Lee, J. K., VanVickle, S. J., Thompson, R. W. (1999) Expression of collagenase-3 (MMP-13) in human abdominal aortic aneurysms and vascular smooth muscle cells in culture. Biochem. Biophys. Res. Commun. 261:904-910.

Nelson, A. R., Fingleton, B., Rothenberg, M. L., Matrisian, L. M. (2000) Matrix metalloproteinases: biologic activity and clinical implications. J. Clin. Oncol. 18:1135-1149.

Nikkola, J., Vihinen, P., Vlaykova, T., Hahka-Kemppinen, M., Kähäri, V.-M., Pyrhönen, S. (2001) High collagenase-1 expression correlates with a favourable chemoimmunotherapy response in human metastatic melanoma. Melanoma Res. 11:157-166.

O'Grady, R. L., Nethery, A., Hunter, N. (1984) A fluorescent screening assay for collagenase using collagen labeled with 2-methoxy-2,4-diphenyl-3(2H)-furanone. Anal. Biochem. 140:490-494.

Pavco, P. A., Bouhana, K. S., Gallegos, A. M., Agrawal, A., Blanchard, K. S., Grimm, S. L., Jensen, K. L., Andrews, L. E., Wincott, F. E., Pitot, P. A., Tressler, R. J., Cushman, C., Reynolds, M. A., Parry, T. J. (2000) Antitumor and antimetastatic activity of ribozymes targeting the messenger RNA of vascular endothelial growth factor receptors. Clin. Cancer Res. 6:2094-2103.

Ravanti, L., Heino, J., López-Otín, C., Kähäri, V. M. (1999a) Induction of collagenase-3 (MMP-13) expression in human skin fibroblasts by three-dimensional collagen is mediated by p38 mitogen-activated protein kinase. J. Biol. Chem. 274:2446-2455.

Ravanti, L., Häkkinen, L., Larjava, H., Saarialho-Kere, U., Foschi, M., Han, J., Kähäri, V.-M. (1999b) Transforming growth factor-β induces collagenase-3 expression by human gingival fibroblasts via p38 mitogen-activated protein kinase. J. Biol. Chem. 274:37292-37300.

Ravanti, L., Toriseva, M., Penttinen, R., Crombleholme, T., Foschi, M., Han, J., Kähäri, V.-M. (2001) Human collagenase-3 expression by fetal skin fibroblasts is induced by transforming growth factor-β via p38 mitogen-activated protein kinase. FASEB J. in press

Reboul, P., Pelletier, J.-P., Tardif, G., Cloutier, J.-M., Martel-Pelletier, J. (1996) The new collagenase, collagenase-3, is expressed and synthesized by human chondrocytes but not by synoviocytes. A role in osteoarthritis. J. Clin. Invest. 97:2011-2019.

Santiago, F. S., Khachigian, L. M. (2001) Nucleic acid based strategies as potential therapeutic tools: mechanistic considerations and implications to restenosis. J. Mol. Med. 79:695-706.

Scanlon, K. J., Ohta, Y., Ishida, H., Kijima, H., Ohkawa, T., Kaminski, A., Tsai, J., Homg, G., Kashani-Sabet, M. (1995) Oligonucleotide-mediated modulation of mammalian gene expression. Faseb J. 9:1288-1296.

Servomaa, K., Kiuru, A., Grénman, R., Pekkola-Heino, K., Pulkkinen, J. O., Rytömaa, T. (1996) p53 mutations associated with increased sensitivity to ionizing radiation in human head and neck cancer cell lines. Cell Prolif. 29:219-30.

Sukhova, G. K., Schönbeck, U., Rabkin, E., Schoen, F. J., Poole, A. R., Billinghurst, R. C., Libby, P. (1999) Evidence for increased collagenolysis by interstitial collagenases-1 and -3 in vulnerable human atheromatous plaques. Circulation 99:2503-2509.

Uitto, V.-J., Airola, K., Vaalamo, M., Johansson, N., Putnins, E. E., Firth, J. D., Salonen, J., López-Otín, C., Saarialho-Kere, U., Kähäri, V.-M. (1998) Collagenase-3 (matrix metalloproteinase-13) expression is induced in oral mucosal epithelium during chronic inflammation. Am. J. Path. 152:1489-1499.

Uría, J. A., Balbín, M., López, J. M., Alvarez, J., Vizoso, F., Takigawa, M., López-Otín, C. (1998) Collagenase-3 (MMP-13) expression in chondrosarcoma cells and its regulation by basic fibroblast growth factor. Am. J. Pathol. 153:91-101.

Usman, N., Blatt, L. M. (2000) Nuclease-resistant synthetic ribozymes: developing a new class of therapeutics. J. Clin. Invest. 106:1197-1202.

Vaalamo, M., Karjalainen-Lindsberg, M.-L., Puolakkainen, P., Kere, J., Saarialho-Kere, U. (1998) Distinct expression profiles of stromelysin-2 (MMP-10), collagenase-3 (MMP-13), macrophagemetalloelastase (MMP-12), and tissue inhibitor of metalloproteinases-3 (TIMP-3) in intestinal ulcerations. Am. J. Pathol. 152:1005-1014.

Vaalamo, M., Mattila, L., Johansson, N., Kariniemi, A.-L., Karjalainen-Lindsberg, M.-L., Kähäri, V.-M., Saarialho-Kere, U. (1997) Distinct populations of stromal cells express collagenase-3 (MMP-13) and collagenase-1(MMP-1) in chronic ulcers but not in normally healing wounds. J. Invest. Dermatol. 109:96-101.

Westermarck, J., Kähäri, V.-M. (1999) Regulation of matrix metalloproteinase expression in tumor invasion. FASEB J. 13:781-92.

Wilkinson, G. W., Akrigg, A. (1992) Constitutive and enhanced expression from the CMV major IE promoter in a defective adenovirus vector. Nucleic Acids Res. 20:2233-2239.

1. An enzymatic RNA molecule which is capable of specifically cleaving a target RNA molecule, which is matrix metalloproteinase 13 (MMP-13) (or collagenase-3) messenger RNA.

2. The RNA molecule according to claim 1, which comprises a hammerhead motif and which is capable of specifically cleaving the target RNA after any sequence UH in said target RNA, where U is a uridine nucleotide and H is an adenosine nucleotide, a cytidine nucleotide or a uridine nucleotide.

3. The RNA molecule according to claim 2, which is capable of specifically cleaving the target RNA after any GUC-sequence in said target RNA.

4. The RNA molecule according to claim 1, which comprises a hammerhead motif and comprises two nucleotide sequences complementary to two nucleotide sequences of the target RNA, located on both sides of the cleavage site in the target RNA, and a catalytic cleaving sequence.

5. The RNA molecule according to claim 4 wherein the first complementary nucleotide sequence is 5′-GUGGUCAA-3′ and the second complementary nucleotide sequence is 5′-ACCUAAGGA-3′ and wherein the catalytic cleaving sequence forms a first catalytic ribonucleotide sequence CUGAUGA and a second catalytic ribonucleotide sequence AAAG, said catalytic ribonucleotide sequences being bound to a separate complementary nucleotide sequence and to a nucleotide sequence capable of base pairing inter se.

6. The RNA molecule according to claim 1, which is not longer than 60 nucleotides.

7. The RNA molecule according to claim 1, which is the antisense ribozyme disclosed in FIG. 1 A.

8. The RNA molecule according to claim 1, which comprises a hairpin motif, a hepatitis delta virus motif, RNaseP RNA or Neurospora VS RNA.

9. The RNA molecule according to claim 1, which comprises a hairpin motif, and which is capable of specifically cleaving the target RNA after any sequence BNGUC in said target RNA, where B is a cytosine nucleotide, a guanosine nucleotide or a uridine nucleotide; N is any nucleotide and G is a guanosine nucleotide, U is a uridine nucleotide and C is a cytidine nucleotide.

10. The RNA molecule according to claim 4, wherein some or all of the ribonucleotides in the complementary chains have modifications in the 2′-OH groups of their ribose units and/or modifications in their internucleotidic phosphodiester linkages and/or said RNA molecule has an inverted 3′-3′-deoxyabasic sugar added to its 3′-end.

11. The RNA molecule according to claim 10, wherein the 2′-OH group in the ribose unit of at least one of the ribonucleotides in the catalytic cleaving sequence is modified.

12. The RNA molecule according to claim 11, wherein the 2′-OH groups in the complementary nucleotide sequences are replaced by 2′-O-methyl, the 2′-OH group(s) in the catalytic cleaving nucleotide sequence is replaced by 2′-O-allyl, and the intemucleotide phoshodiester linkage in the complementary sequences are replaced by phosphorothioate linkages.

13. The RNA molecule according to claim 7, wherein some or all of the ribonucleotides in the complementary chains have modifications in the 2′-OH groups of their ribose units and/or modifications in their intemucleotidic phosphodiester linkages, and/or said RNA molecule has an inverted 3′-3′-deoxyabasic sugar added to its 3′-end.

14. The RNA molecule according to claim 13, wherein the 2′-OH group in the ribose unit of at least one of the ribonucleotides in the catalytic cleaving sequence is modified.

15. The RNA molecule according to claim 14, wherein the 2′-OH groups in the complementary nucleotide sequences are replaced by 2′-O-methyl, the 2′-OH group(s) in the catalytic cleaving nucleotide sequence is replaced by 2′-O-allyl, and the internucleotide phoshodiester linkage in the complementary sequences are replaced by phosphorothioate linkages.

16. The RNA molecule according to claim 1, which comprises a hairpin motif, wherein some or all of the ribonucleotides in its complementary chains have modifications in the 2′-OH groups of their ribose units and/or modifications in their internucleotidic phosphodiester linkages and/or said RNA molecule has an inverted 3′-3′-deoxyabasic sugar added to its 3′-end.

17. A pharmaceutical composition comprising a therapeutically effective amount of an RNA molecule according to any of the claims 1 to 16 in a pharmaceutically acceptable carrier.

18. A pharmaceutical composition according to claim 17, wherein the RNA molecule is complexed with a cationic lipid, packed in a liposome, incorporated in a cyclodextrin, a bioresorbable polymer or other suitable carrier for slow release administration, a nanoparticle or a hydrogel.

19. An isolated mammalian cell including an RNA molecule according to any of the claims 1 to 16.

20. An expression vector including nucleic acid encoding the enzymatic RNA according to any of the claims 1 to 9, in a manner which allows expression of said enzymatic RNA within a mammalian cell.

21. The expression vector according to claim 20, wherein the nucleic acid encoding the enzymatic RNA is inserted in a DNA sequence.

22. The expression vector according to claim 20, wherein the nucleic acid encoding the enzymatic RNA is inserted in a viral vector.

23. The expression vector according to claim 22, wherein the viral vector is based on an adenovirus, an alphavirus, an adeno-associated virus, a retrovirus or a herpes virus.

24. A pharmaceutical composition comprising an expression vector including nucleic acid encoding the enzymatic RNA according to any of the claims 1 to 9, in a manner which allows expression of said enzymatic RNA within a mammalian cell, and a pharmaceutically acceptable carrier.

25. The pharmaceutical composition according to claim 24, wherein the expression vector is complexed with a cationic lipid, packed in a liposome, incorporated in a cyclodextrin, a bioresorbable polymer or other suitable carrier for slow release administration, a nanoparticle or a hydrogel.

26. A method for reducing or eliminating the expression of MMP-13 in an individual, said method comprising administering to said individual
i) an effective amount of an enzymatic RNA according to any of the claims 1-16, or
ii) an expression vector including nucleic acid encoding the enzymatic RNA according to any of the claims 1 to 9, in a manner which allows expression of said enzymatic RNA within a mammalian cell.

27. A method for treating or preventing cancer, or preventing or inhibiting cancer growth, invasion or metastasis in an individual, said method comprising administering to said individual
i) an effective amount of an enzymatic RNA according to any of the claims 1-16, or
ii) an expression vector including nucleic acid encoding the enzymatic RNA according to any of the claims 1 to 9, in a manner which allows expression of said enzymatic RNA within a mammalian cell.

28. The method according to claim 27, wherein cancer is treated or prevented by
i) suppressing invasion of cancer cells, and/or
ii) inhibiting tumor growth, and/or
iii) inducing cancer cell apoptosis.

29. The method according to claim 27, wherein said method is used as an adjuvant therapy.

30. A method for inducing of cancer cell apoptosis in an individual, said method comprising inhibiting the expression or inhibiting or suppressing the activity of MMP-13 in said individual.

31. The method according to claim 30, wherein said individual is treated with a small molecule MMP-13 inhibitor, an intracellular or extracellular activity blocking antibody, an MMP-13 mRNA antisense oligonucleotide, a short interfering RNA or a ribozyme.

32. A method for treating or preventing of an inflammatory condition, especially osteoarthritis, rheumatoid arthritis, rupture of atherosclerotic plaque, aorta aneurysm, congestive hearth failure, chronic skin wounds, gastrointestinal ulcer, or chronic periodontitis or gingivitis in an individual, said method comprising administering to said individual
i) an effective amount of an enzymatic RNA according to any of the claims 1-16, or
ii) an expression vector including nucleic acid encoding the enzymatic RNA according to any of the claims 1 to 9, in a manner which allows expression of said enzymatic RNA within a mammalian cell.

33. A method for detecting or quantifying the level of MMP-13 in a tissue or fluid by
i) determining the MMP-13 mRNA expression from said tissue or body fluid by RT-PCR, or by a hybridizing technique, or
ii) subjecting the tissue or body fluid expected to contain the protein MMP-13 to an antibody recognizing MMP-13, and detecting and/or quantifying said antibody, or subjecting said tissue or body fluid to analysis by proteomics technique.

34. A method for diagnosing an MMP-13 related cancer or MMP-13 related inflammatory condition, especially osteoarthritis, rheumatoid arthritis, rupture of atherosclerotic plaque, aorta aneurysm, congestive hearth failure, chronic skin wounds, gastrointestinal ulcer, or chronic periodontitis or gingivitis in an individual, comprising subjecting a tissue or body fluid sample from said individual to a method according to claim 33, for detecting or quantifying the level of MMP-13 in said sample.
